(19) United States
(12) Patent Application Publication
(10) Pub. No.: US 2021/0347827 A1
(43) Pub. Date: Nov. 11, 2021

(54) LINEAR POLYFUNCTIONAL MULTIMER BIOMOLECULE COUPLED TO POLYUBIQUITIN LINKER AND USE THEREOF

(71) Applicant: ONEGENE BIOTECHNOLOGY INC., Gyeonggi-do (KR)

(72) Inventors: Sungjin Park, Gyeonggi-do (KR); Dae Seong Im, Gyeonggi-do (KR); Jaeyoung Choi, Gyeonggi-do (KR)

(73) Assignee: ONEGENE BIOTECHNOLOGY INC., Gyeonggi-do (KR)

(21) Appl. No.: 15/733,814

(22) PCT Filed: May 28, 2019
(86) PCT No.: PCT/KR19/06376

§ 371 Date: Nov. 30, 2020

(30) Foreign Application Priority Data
May 28, 2018 (KR) 10-2018-0060502

(51) Int. Cl. C07K 14/00; C07K 14/47; C12N 9/00; C12N 9/10

(52) U.S. Cl. CPC C07K 14/001; C07K 14/4702; C12N 9/93; C12N 9/104; C07K 2319/95; C12Y 602/01; C07K 2319/21; C07K 2319/23; C12Y 603/02019

(57) ABSTRACT

The present invention provides a linear multimeric biomolecule polymer wherein a biomolecule is bonded to a polyubiquitin scaffold formed of two or more covalently bonded ubiquitins, by obtaining, from a host cell, a biomolecule bonded with a ubiquitin C-terminal tag through recombinant expression, and polyubiquitinating the biomolecule in vitro in the presence of proteins involved in ubiquitination, E1 (activation enzyme), E2 (conjugation enzyme), and E3 (ligase), and a substrate. The polymer according to the present invention may be used in the separation and purification of a biomolecule, the separation of a target material that binds to the biomolecule, etc.

### CROSS-REFERENCE TO RELATED APPLICATIONS

This application is a § 371 national-stage application based on International Application No. PCT/KR19/06376, filed on May 28, 2019, which claims priority from Application 10-2018-0060502, filed on May 28, 2018, in the Republic of Korea, the contents of each are incorporated herein by reference in their entirety.

### SEQUENCE LISTING

The instant application contains a Sequence Listing which has been submitted electronically in ASCII format and is hereby incorporated by reference in its entirety. Said ASCII copy, created on Jul. 6, 2021, is named CPH-00501_SL.txt and is 42,578 bytes in size.

### TECHNICAL FIELD

The present invention relates to a method for preparing a biomolecule including a protein into a polymer in a multimeric form. Specifically, the present invention relates to a method for preparing a biomolecule recombinantly expressed from a host cell into a linear polyfunctional multimeric biomolecule polymer using a ubiquitination system.

### BACKGROUND ART

Preparing biomolecules and/or small molecule chemical compounds including proteins, peptides, polypeptides, antibodies, DNA and RNA in multimeric form has various advantages. For example, the physicochemical properties of protein such as solubility, gelation, thermal stability and pH stability can be improved by linking two or more homogeneous or heterogeneous proteins using a fusion or a cross linker (or a cross-linking agent). For example, CLEA (cross-linked enzyme aggregate), laccase formed by multiple linking through a cross linker showed more enhanced stability and performance during starch oxidation, and CLEA of another enzyme, nitrile hydratase showed an excellent increase in activity in the conversion of acrylonitrile to acrylamide, and did not lose activity during 36 recycles. In addition, many proteins form complexes in cells to perform complex functions, which are known to be due to the proximity effect of proteins. For example, the cellulase (Novozymes Cellic® CTec3), which is produced by preparing enzymes necessary for lignocellulose degradation, such as, cellulase, beta glucosidase (β-glucosidase), hemicellulase and the like in the form of a complex mixture using a scaffold, is known to exhibit a 3.5-fold or more increased effect in the degradation of lignocellulose. In addition, such a protein in the multimeric form exhibits a channeling effect. That is, if enzymes involved in a coupled reaction are present adjacent to each other, the transfer of the intermediate is efficient and the efficiency of the entire reaction is greatly increased. In addition, it is proposed to be desirable for an increase in its efficiency to use a homogeneous or heterogeneous protein in a multimeric form when analyzing an any substance using a protein immobilized on a bead or a substrate, or separating and/or purifying a substance to be detected. As described above, although the protein in the multimeric form provides various advantages in industrial and medical applications, it has been known that it is difficult to fabricate a protein having such a structure. For example, there is a method of developing and producing a multimeric protein as a new fusion enzyme by designing in-frame at the genetic stage. However, since a new protein must be designed and produced, it takes a long time to develop it and it is difficult to fuse two or more enzymes in reality. In addition, in the case of a method of fabricating a protein multimer construct (CLEA) using a chemical cross linker, the activity may be inhibited because a chemical bond does not occur at a specific site but can occur anywhere on the protein surface. Proteins that form a multimer construct must be capable of being prepared through synthesis or microbial expression, and the active sites of these proteins must not be disturbed.

A method of using ubiquitin has been proposed as a method for separating and/or purifying a protein of interest. It is the method in which first, a gene encoding a protein bound to ubiquitin is expressed in prokaryotic cells to prepare a fusion protein linked to ubiquitin, and then treated with ubiquitin cleavage enzyme to effectively separate and purify only the protein of interest from the ubiquitin fusion protein. U.S. patent application Ser. No. 10/504,785 relates to the expression of a recombinant gene and the purification of the expressed protein, and it describes that the fusion protein is prepared in which the nucleotide encoding the C-terminal domain of the ubiquitin-like protein (Ubl) is operatively bound to the nucleotide encoding the protein of interest, and it is expressed in a host cell. Korean Patent Application No. 10-2005-0050824 describes the use of ubiquitin as a fusion partner in expressing a recombinant protein. In addition, Korean Patent Application No. 10-2015-0120852 relates to the use of an ubiquitin column for purifying a protein, and describes that a polyubiquitin chain is loaded on the column, and the protein is purified using in vitro ubiquitination including E2. In addition, U.S. patent application Ser. No. 12/249,334 is to solve the problem of water solubility and folding, which is a problem in preparing by expressing a recombinant protein, and describes the use of SUMO having a cleavage site recognized by Ulp1 protease (Ubl-specific protease 1) for facilitating expression, separation and purification of the recombinant protein, and for increasing the activity of the protein. However, these methods only describe the use of ubiquitin for protein expression, and do not describe or suggest the production of a protein in a multimeric form, and since the protein to be separated and purified randomly binds to ubiquitin, these methods still have a limit to separation or analysis efficiency.

Accordingly, the present inventors have made ceaseless efforts to develop a method for preparing a protein in a multimeric form having a high degree of integration without inhibiting the activity of the protein. As a result, a biomolecule bound to ubiquitin was recombinantly expressed from a host cell and was reacted in vitro with an enzyme related to ubiquitination to form a linear polyfunctional multimeric biomolecule polymer bound to a polyubiquitin scaffold. Based on the above, the present inventors completed the present invention.

### DETAILED DESCRIPTION OF THE INVENTION

### Technical Problem

As described above, an object of the present invention is to provide a method of immobilizing or cross-linking biomolecules such as proteins in vitro using a Ubiquitin C-terminal Tag (UCT).

Another object of the present invention is to provide a linear polyfunctional multimeric biomolecule in which a target biomolecule is bound to a polyubiquitin scaffold and a method for preparing the same.

Another object of the present invention is to provide a construct in which the linear polyfunctional multimeric biomolecule is immobilized and a method for preparing the same.

Another object of the present invention is to provide a method for separating and purifying a target material using the linear polyfunctional multimeric biomolecule.

Another object of the present invention is to provide a method of analyzing, or separating and purifying a target material that binds to the biomolecule using the linear polyfunctional multimeric biomolecule.

Another object of the present invention is to provide a method for site-specifically binding two or more biomolecules and/or small molecule chemical compounds using polyubiquitin as a linker.

Another object of the present invention is to provide the use of polyubiquitin for site-specifical binding of two or more biomolecules and/or small molecule chemical compounds.

Another object of the present invention is to provide a pharmaceutical composition comprising the linear polyfunctional multimeric biomolecule of the present invention.

### Solution to Problem

In order to achieve the above objects, the present invention provides a method for preparing a linear polyfunctional multimeric biomolecule, wherein the method comprises (i) recombinantly expressing a biomolecule to which a ubiquitin C-terminal tag is fused or bound by a linker from a host cell including a prokaryotic cell or a eukaryotic cell, and (ii) adding E1, E2 and E3 enzymes for ubiquitination, or E1 and E2 enzymes for ubiquitination to a cell lysate of the host cell and reacting them, wherein the biomolecule is bound to a polyubiquitin scaffold formed of two or more covalently bonded ubiquitins, and the biomolecule comprises two or more binding moieties, each specific for different binding sites. Accordingly, in the present invention, an initiator that initiates the formation of a linear polyfunctional multimeric biomolecule polymer or complex may be E3, E2, E1, a free ubiquitin, or a target substrate of E3. Here, the E2 enzyme may bind to the lysine at the 48th or 63rd amino acid residue of ubiquitin, and the E2 enzyme may be an E2-25K ubiquitin conjugating enzyme, or a ubiquitin conjugating enzyme complex Ucb13-MMS2.

In one embodiment of the present invention related thereto, the recombinantly expressed biomolecule is one in which a C-terminal portion of the glycine at the 76th amino acid residue of a ubiquitin C-terminal tag is extended by 1 to 50 amino acids, and the method may further comprise adding DUB (deubiquitinating enzyme), for example, UH1, YUH2, UCH-L1, UCH-L2 or UCH-L3, to the recombinantly expressed biomolecule before or after the reaction of the above step (ii).

In one embodiment of the present invention related thereto, the biomolecule has active sites that specifically bind to other biomolecules, small molecule chemical compounds or nanoparticles or the like, and the biomolecule may be one or more selected from the group consisting of an enzyme, a protein, a peptide, a polypeptide, an antibody, DNA and RNA, but is not limited thereto, and ATP may be further added to the above step (ii) and reacted with them. Advantageously, each of the enzyme, protein, peptide, polypeptide, antibody, DNA and RNA may be homogeneous or heterogeneous. That is, the monomers constituting the linear polyfunctional multimer complex of the present invention may be homogeneous or heterogeneous proteins, or proteins and peptides or antibodies, respectively, and the monomers of various types of biomolecules may be formed into a linear polyfunctional multimer complex as necessary. Here, the biomolecule may be one or more selected from the group consisting of protein A, protein G, lysin, endolysin, protease, hydrolase, oxidoreductase, lyase, affinity ligand and receptor, but is not limited thereto. In addition, the biomolecule may be one or more selected from the group consisting of insulin, insulin analogue, glucagon, glucagon-like peptides (GLP-1 and the like), GLP-1/glucagon dual agonist, exendin-4, exendin-4 analogue, insulin secreting peptide and an analogue thereof, human growth hormone, growth hormone releasing hormone (GHRH), growth hormone releasing peptide, granulocyte colony stimulating factor (G-CSF), anti-obesity peptide, G-protein-coupled receptor, leptin, GIP (gastric inhibitory polypeptide), interleukins, interleukin receptors, interleukin binding proteins, interferons, interferon receptors, cytokine binding proteins, macrophage activator, macrophage peptide, B cell factor, T cell factor, suppressive factor of allergy, cell necrosis glycoprotein, immunotoxin, lymphotoxin, tumor necrosis factor (TNF), tumor inhibitory factor, metastasis growth factor, alpha-1 antitrypsin, albumin, α-lactalbumin, apolipoprotein-E, erythropoietin (EPO), high glycosylated erythropoietin, angiopoietins, hemoglobin, thrombin, thrombin receptor activating peptide, thrombomodulin, blood factors VII, VIIa, VIII, IX, and XIII, plasminogen activator, fibrin-binding peptide, urokinase, streptokinase, hirudin, protein C, C-reactive protein, renin inhibitor, collagenase inhibitor, superoxide dismutase, platelet derived growth factor, epithelial growth factor, epidermal growth factor, angiostatin, angiotensin, bone formation growth factor, bone formation promoting protein, calcitonin, atriopeptin, cartilage inducing factor, elcatonin, connective tissue activator, tissue factor pathway inhibitor, follicle stimulating hormone (FSH), luteinizing hormone (LH), luteinizing hormone releasing hormone (LHRH), nerve growth factors, parathyroid hormone (PTH), relaxin, secretin, somatomedin, adrenal cortical hormone, cholecystokinin, pancreatic polypeptide, gastrin releasing peptide, corticotropin releasing factor, thyroid stimulating hormone (TSH), autotaxin, lactoferrin, myostatin, receptor, receptor antagonist, fibroblast growth factor, adiponectin, interleukin receptor antagonist, aspartate, 6×His, chitin binding domain, GST, thrombin, FLAG tag, cell surface antigen, virus derived vaccine antigen, monoclonal antibody, polyclonal antibody and antibody fragments, but is not limited thereto.

In one embodiment of the present invention related thereto, E1, E2 and E3 used for in vitro ubiquitination may be selected and used in any combination. For example, if the E2 is UBCH5A (UBE2D1), it may be selected from the group consisting of RSP5 (UniProt ID. P39940), DTX2 (UniProt ID. Q86UW9), DTX3 (UniProt ID. Q8N9I9), MID1 (UniProt ID. 015344), RING1 (UniProt ID. Q06587), RNF11 (UniProt ID. Q9Y3C5), RNF111 (UniProt ID. Q6ZNA4), RNF126 (UniProt ID. Q9BV68), RNF115 (UniProt ID. Q9Y4L5), RNF14 (UniProt ID. Q9UBS8), RNF185 (UniProt ID. Q96GF1), RNF2 (UniProt ID. Q99496), RNF5 (UniProt ID. Q99942), TRAF6 (UniProt ID. Q9Y4K3), TRIM8 (UniProt ID. Q9BZR9), ZNRF1 (UniProt ID. Q8ND25), XIAP (UniProt ID. P98170), and TRIM39 (UniProt ID. Q9HCM9). In addition, if the E2 is UBC7, the E3 may be DOA10 (UniProt ID. P40318), UFD4 (UniProt ID. P33202), HRD1 (UniProt ID. Q08109) or HRD3 (UniProt ID. Q05787), and if the E2 is UBE2W (UBC16), as the E3 interacting with it, MARCH5 (UniProt ID. Q9NX47) or RNF5 (UniProt ID. Q99942) may be used, but is not limited thereto.

In another embodiment of the present invention related thereto, advantageously, the ubiquitin C-terminal tag is one in which the lysine at the 48th or the 63rd amino acid residue from the N-terminus of the ubiquitin is substituted with alanine, or more advantageously, all lysines except for one lysine at any position are deleted or substituted with amino acids other than lysine. In addition, all lysines of the ubiquitin except for the lysine at the 11th, 48th, or 63rd amino acid residue starting from the N-terminal Met1 of the ubiquitin may be substituted with arginine. In addition, the ubiquitin C-terminal tag may be one in which two or more ubiquitins may be repeatedly linked in a head-to-tail form. In this case, the ubiquitin linked in the head-to-tail form may be one in which the glycines at the 75th and 76th amino acid residue from the N-terminus may be substituted with other amino acids including valine, or the leucine at the 73rd amino acid residue may be substituted with proline.

In another embodiment of the present invention related thereto, the reaction of the above step (ii) is carried out in the presence of a substrate or substrates or a bead for immobilization of ubiquitin, and E3 ligase may be attached to one terminus of the biomolecule.

In addition, the present invention provides a linear multimeric biomolecule polymer comprised of a polyubiquitin scaffold and a biomolecule, wherein the polyubiquitin scaffold is formed by linking two or more ubiquitins, for example, through covalent bonds, and the biomolecule is each bound to the ubiquitin. The biomolecule is preferably each bound to the N-terminus of the ubiquitin. The initiator that initiates the formation of a linear multimeric biomolecule polymer may be E3, E2, E1, a free ubiquitin, or a substrate. In addition, the linear multimeric biomolecule polymer may be comprised of 2 to 20 biomolecules.

In one embodiment of the present invention related thereto, the biomolecule may be one or more selected from the group consisting of an enzyme, a protein, a peptide, a polypeptide, an antibody, DNA and RNA, miRNA, siRNA, and a small molecule chemical compound. Advantageously, each of the protein, peptide, polypeptide, antibody, DNA and RNA may be of different types.

In another embodiment of the present invention related thereto, the linear polyfunctional multimeric biomolecule complex or polymer may be originated from E3, E2, E1, a free ubiquitin, or a substrate, and the E3 may be Rsp5, WWP1, nedd4 or XIAP, or a minimal catalytic domain thereof, and the E2 may be Ubc7, Ubch5a, E2-25K (GenBank ID-U58522.1), Ubc13-MMS2 (Unipot ID-P52490) complex, or a minimal catalytic domain thereof.

In another embodiment of the present invention related thereto, the ubiquitin is one in which the lysine at the 48th or the 63rd amino acid residue from the N-terminus of the ubiquitin is substituted with alanine, or more advantageously, other lysines except for one lysine at any position thereof are deleted or substituted with amino acids other than lysine. In addition, the free ubiquitin may be one in which other lysines except for one lysine at any position thereof are deleted or substituted with amino acids other than lysine, and it may be one in which all lysines except for the lysine at the 11th, 48th, or 63rd amino acid residue from the N-terminus are substituted with arginine. In addition, the free ubiquitin may be one in which a C-terminal portion of the glycine at the 76th amino acid residue from the N-terminus thereof may be extended by 1 to 50 amino acids, and the amino acid may be aspartate, 6×His tag, or GST tag, and the protein may have PPPY for Rsp5 or Nedd4-1,2. The biomolecule may be linked to the N-terminal Met1 of the free ubiquitin, and an initiator, for example, E3, E2, E1, a free ubiquitin or a substrate may be attached to one terminus of the biomolecule. In addition, the substrate may be a protein that comprises an amino acid sequence that recognizes E3 ligase and comprises one or more lysine to which ubiquitin is capable of binding.

In another embodiment of the present invention related thereto, the present invention also provides a method of separating a biomolecule expressed in a host cell in vitro, wherein the method comprises (i) recombinantly expressing a biomolecule to which a ubiquitin C-terminal tag is fused or bound by a linker from a host cell including a prokaryotic cell, a eukaryotic cell, or an animal cell; (ii) adding E1, E2 and E3 proteins for ubiquitination to a cell lysate of the host cell and reacting them to form a linear polyfunctional multimeric biomolecule complex in which a biomolecule to be separated and purified is bound to a polyubiquitin scaffold formed of two or more covalently bonded ubiquitins; and (ii) separating the linear multimeric biomolecule complex.

In one embodiment of the present invention related thereto, the biomolecule is one or more selected from the group consisting of a protein, a peptide, a polypeptide, an antibody, DNA and RNA, and ATP is further added to the above step (ii) and reacted. Advantageously, each of the protein, peptide, polypeptide, antibody, DNA and RNA may be of different types.

In another embodiment of the present invention related thereto, advantageously, the ubiquitin C-terminal tag is one in which the lysine at the 48th or the 63rd amino acid residue thereof is substituted with alanine, or more advantageously, all lysines except for one lysine at any position are deleted or substituted with amino acids other than lysine.

In another embodiment of the present invention related thereto, the reaction of the above step (ii) is carried out in the presence of a substrate or substrates or a bead for immobilization of ubiquitin, and E3 ligase may be attached to one terminus of the biomolecule.

The present invention provides a separation column comprising a linear polyfunctional multimeric biomolecule complex. In one embodiment related thereto, the biomolecule may be a protein or an antibody. In the present invention, the linker may be a peptide consisting of 3 to 50 amino acids, but is not limited thereto.

In another embodiment of the present invention, the present invention provides a linear polyfunctional multimeric biomolecule polymer comprised of a polyubiquitin scaffold and a biomolecule, wherein the linear polyfunctional multimeric biomolecule polymer comprises two or more binding moieties that are specific for different binding sites, and the polyubiquitin scaffold is formed of two or more covalently bonded ubiquitins; the biomolecule has active sites that specifically bind to other biomolecules, small molecule chemical compounds or nanoparticles or the like, and the biomolecule is bound to the N-terminus, the C-terminus, or both the N-terminus and the C-terminus of the ubiquitin. Here, the biomolecule polymer may be comprised of 2 to 4 biomolecules, and the biomolecule may be bound by a linker to the N-terminus, the C-terminus, or both the N-terminus and the C-terminus of the ubiquitin. the linker may be a combination of 1 to 6 repeats of GGGGS or EAAAK. In this case, the biomolecule bound to the N-terminus of the ubiquitin is the distal end of the linear multimeric biomolecule polymer, and the biomolecule bound to the C-terminus or the N-terminus or both the C-terminus and the N-terminus of the ubiquitin is the proximal end of the linear multimeric biomolecule polymer. In other embodiment related thereto, the polyubiquitin scaffold may be formed by covalently linking a donor ubiquitin in which all lysines of the ubiquitin are substituted with arginine, and an acceptor ubiquitin in which all lysines of the ubiquitin except for the lysine at the 11th, 48th, or 63rd amino acid residue are substituted with arginine, and the leucine at the 73rd amino acid residue from the N-terminus of the ubiquitin may be substituted with proline.

In other embodiment related thereto, the linear polyfunctional multimeric biomolecule polymer may be comprised of 2 to 20 biomolecules.

In other embodiment related thereto, the biomolecule may be one or more selected from the group consisting of an enzyme, a protein, a peptide, a polypeptide, an antibody, an antibody fragment, DNA and RNA, and the biomolecule may be one or more selected from the group consisting of protein A, protein G, lysin, endolysin, protease, hydrolase, oxidoreductase, lyase, affinity ligand and receptor. In addition, the biomolecule may be selected from the group consisting of insulin, insulin analogue, glucagon, glucagon-like peptides (GLP-1 and the like), GLP-1/glucagon dual agonist, exendin-4, exendin-4 analogue, insulin secreting peptide and an analogue thereof, human growth hormone, growth hormone releasing hormone (GHRH), growth hormone releasing peptide, granulocyte colony stimulating factor (G-CSF), anti-obesity peptide, G-protein-coupled receptor, leptin, GIP (gastric inhibitory polypeptide), interleukins, interleukin receptors, interleukin binding proteins, interferons, interferon receptors, cytokine binding proteins, macrophage activator, macrophage peptide, B cell factor, T cell factor, suppressive factor of allergy, cell necrosis glycoprotein, immunotoxin, lymphotoxin, tumor necrosis factor (TNF), tumor inhibitory factor, metastasis growth factor, alpha-1 antitrypsin, albumin, α-lactalbumin, apolipoprotein-E, erythropoietin (EPO), high glycosylated erythropoietin, angiopoietins, hemoglobin, thrombin, thrombin receptor activating peptide, thrombomodulin, blood factors VII, VIIa, VIII, IX, and XIII, plasminogen activator, fibrin-binding peptide, urokinase, streptokinase, hirudin, protein C, C-reactive protein, renin inhibitor, collagenase inhibitor, superoxide dismutase, platelet derived growth factor, epithelial growth factor, epidermal growth factor, angiostatin, angiotensin, bone formation growth factor, bone formation promoting protein, calcitonin, atriopeptin, cartilage inducing factor, elcatonin, connective tissue activator, tissue factor pathway inhibitor, follicle stimulating hormone (FSH), luteinizing hormone (LH), luteinizing hormone releasing hormone (LHRH), nerve growth factors, parathyroid hormone (PTH), relaxin, secretin, somatomedin, adrenal cortical hormone, cholecystokinin, pancreatic polypeptide, gastrin releasing peptide, corticotropin releasing factor, thyroid stimulating hormone (TSH), autotaxin, lactoferrin, myostatin, receptor, receptor antagonist, fibroblast growth factor, adiponectin, interleukin receptor antagonist, cell surface antigen, virus derived vaccine antigen, monoclonal antibody, polyclonal antibody and antibody fragments.

In other embodiment related thereto, the linear polyfunctional multimeric biomolecule polymer may be originated from E3, E2, E1, a free ubiquitin, or a substrate, and the E3 may be Rsp5, WWP1, nedd4 or XIAP, or a minimal catalytic domain thereof, and the E2 may be Ubc7, Ubch5a, E2-25K, Ubc13-MMS2 complex, or a minimal catalytic domain thereof. Here, the free ubiquitin may be one in which other lysines except for one lysine at any position thereof are deleted or substituted with amino acids other than lysine. In addition, the free ubiquitin may be one in which all lysines except for the lysine at the 11th, 48th, or 63rd amino acid residue from the N-terminus thereof are substituted with arginine, and the free ubiquitin may be one in which a C-terminal portion of the glycine at the 76th amino acid residue from the N-terminus thereof is extended by 1 to 50 amino acids, or the free ubiquitin may be extended by aspartate, 6×His tag, or GST tag, or the biomolecule may be linked to the N-terminal Met1 of the free ubiquitin. In other embodiment related thereto, E3, E2, E1, a free ubiquitin or a substrate may be attached to one terminus of the biomolecule as an initiator, and the substrate may be a protein that comprises an amino acid sequence that recognizes E3 ligase and comprises one or more lysine to which ubiquitin is capable of binding, and the protein may comprise PPPY for Rsp5 or Nedd4-1,2. In other embodiment related thereto, the ubiquitin may be one in which other lysines except for one lysine at any position thereof are deleted or substituted with amino acids other than lysine.

In another aspect of the present invention, herein is provided a pharmaceutical composition, wherein the composition comprises the linear polyfunctional multimeric biomolecule of the present invention and a pharmaceutically acceptable carrier, and has an increased in vivo stability. The pharmaceutical composition according to the present invention may be administered through various routes, for example, orally, transdermally, subcutaneously, intravenously or intramuscularly into the body. In addition, the pharmaceutical composition according to the present invention may be formulated using a method well known in the art. The formulation may be in the form of a tablet, pill, powder, sachet, elixir, suspension, emulsion, solution, syrup, aerosol, soft or hard gelatin capsule, sterile injectable solution, sterile powder, and the like. Examples of suitable carriers, excipients and diluents for formulation include lactose, dextrose, sucrose, sorbitol, mannitol, xylitol, erythritol, maltitol, starch, gum acacia, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate, mineral oil, and the like, which may be used. The formulation may further comprise fillers, anticoagulants, lubricants, wetting agents, flavoring agents, emulsifying agents, preservatives, and the like. The actual dosage of the biomolecule of the present invention is determined depending on the type of the physiologically active polypeptide as an active ingredient, along with various related factors such as the disease to be treated, the route of administration, the age, sex and weight of the patient, and the severity of the disease.

The term “polyfunctional” used in the present invention is used interchangeably with “multifunctional” or “multivalent,” and is understood to have the same meaning in the present invention.

The term “polyfunctional multimeric biomolecule” used in the present invention is defined as a biomolecule comprising two or more binding moieties, each specific for different binding sites.

The terms “complex,” “polymer,” “conjugate,” and “construct” used in the present invention are used interchangeably in the present specification as having the same meaning when these terms refer to the linear polyfunctional multimeric biomolecule of the present invention bound to polyubiquitin by a linker.

The term “ubiquitin C-terminal tag” used in the present invention may be understood as a ubiquitin forming the initiating portion in the formation of a polyubiquitin.

### Effect of the Invention

According to the present invention, since the linkage between linear polyfunctional multimeric biomolecule polymers or complexes is made by UCT, polyubiquitin formed by linkage of UCT may act as a rigid scaffold or linker that maintains the spacing and orientation between biomolecules bound to UCT. In addition, since the enzymatic reaction (E1-E2-E3) is used for the binding between UCTs, the biomolecule-UCT expressed in a host cell can be easily used in the form of a cell lysate mixture without separate process steps of separation and purification. In addition, the biomolecule of the present invention may be one or more selected from the group consisting of a protein, a peptide, a polypeptide, an antibody, an antibody fragment, DNA and RNA, and for example, a heterogeneous protein may be used to give the modularized functionality to a linear polyfunctional multimer polymer. Enzymes made from linear polymers by enzyme proximity effects exhibit a more enhanced effect in catalytic functionality and stability. When heterogeneous enzymes constituting a coupled reaction are made into a linear multimer construct, a synergistic effect is achieved in that the overall reaction efficiency is higher than that of a bulk mixture due to a channeling effect. In addition, since the target biomolecule-UCT can be attached to the stationary phase using an enzymatic reaction, the biomolecule-UCT does not need to be purified and separated purely. Therefore, a linear multimer construct is synthesized and immobilized in a crude-mixture comprising a biomolecule-UCT, such as cell lysates or culture media. Therefore, the immobilized enzyme can be produced economically.

### BRIEF DESCRIPTION OF DRAWINGS

FIG. 1 schematically shows the process of preparing the linear polyfunctional multimer fusion protein (Ubstac) of the present invention.

FIG. 2 shows results of confirming the UCT fusion protein in a multimeric form formed by the Ubstac reaction of the present invention.

FIG. 3 shows results of confirming the UCT fusion protein in a multimeric form formed by the Ubstac reaction of the present invention.

FIG. 4 schematically shows the preparation of the linear polyfunctional multimer fusion protein of the present invention and the use thereof by immobilization.

FIG. 5 shows the results of Ubstac preparation using only E1-E2.

FIG. 6 schematically shows the preparation of the linear polyfunctional multimer fusion protein of the present invention and the use thereof by immobilization.

FIG. 7 schematically shows the head-to-tail UCT and UbStac method.

FIG. 8 is a result of confirming by SDS-PAGE after purification of xylose reductase (XR) prepared according to the present invention by GPC.

FIG. 9 is a result of confirming by SDS-PAGE after purification of oxaloacetate decarboxylase (OAC) prepared according to the present invention by GPC.

FIG. 10 is a result of confirming by SDS-PAGE after purification of xylitol dehydrogenase (XDH) prepared according to the present invention by GPC.

FIG. 11 is a result of confirming by SDS-PAGE after purification of triosephosphate isomerase (TIM) prepared according to the present invention by GPC.

FIG. 12 is a result of confirming by SDS-PAGE after purification of aldolase (ALD) prepared according to the present invention by GPC.

FIG. 13 is a result of confirming by SDS-PAGE after purification of fructose 1,6-bisphosphatase (FBP) prepared according to the present invention by GPC.

FIG. 14 is a result of confirming by SDS-PAGE after purification of pyruvate oxidase (POPG) prepared according to the present invention by GPC.

FIG. 15 is a result of analysis of the activity of xylose reductase.

FIG. 16 is a result of analysis of the stability of xylose reductase.

FIG. 17 is a result of analysis of the activity of oxaloacetate decarboxylase.

FIG. 18 is a result of analysis of the stability of oxaloacetate decarboxylase.

FIG. 19 is a result of analysis of the activity of xylitol dehydrogenase.

FIG. 20 is a result of analysis of the stability of xylitol dehydrogenase.

FIG. 21 is a result of analysis of the activity of pyruvate oxidase.

FIG. 22 shows a result of the Ubstac of the structure to which three enzymes, TIM, ALD and FBP are bound.

FIG. 23 shows the synergistic effect by TIM, ALD and FBP enzymes.

FIG. 24 is a result of preparing and confirming Protein A and Protein G linear polyfunctional multimer complexes.

FIG. 25 is a result of preparing and confirming hGH in which aspartate is extended at the C-terminal portion of the glycine at amino acid 76 of the ubiquitin C-terminal tag.

FIG. 26 is a result of preparing and confirming the polymer originated from E3.

FIG. 27 is a result of preparing and confirming the polymer of hGH expressed in an extended form with aspartate at the C-terminus of a UCT repeatedly linked in a head-to-tail form.

FIG. 28 shows that the binding activity of human derived IgG to the beads on which the Protein A polymer is immobilized is increased compared to the beads on which the Protein A monomer is immobilized.

FIG. 29 schematically shows the structure of a linear polyfunctional multimeric biomolecule polymer bound to the N-terminus, the C-terminus, or both the N-terminus and the C-terminus of the ubiquitin, respectively.

### BEST MODE FOR CARRYING OUT THE INVENTION

In an embodiment, the present invention provides a method for preparing a linear polyfunctional multimeric biomolecule, wherein the method comprises (i) recombinantly expressing a biomolecule to which a ubiquitin C-terminal tag is fused or bound by a linker from a host cell including a prokaryotic cell or a eukaryotic cell, and (ii) adding E1, E2 and E3 enzymes for ubiquitination, or E1 and E2 enzymes for ubiquitination to a cell lysate of the host cell and reacting them, wherein the biomolecule is bound to a polyubiquitin scaffold formed of two or more covalently bonded ubiquitins, and the biomolecule comprises two or more binding moieties, each specific for different binding sites. Accordingly, in the present invention, an initiator that initiates the formation of a linear polyfunctional multimeric biomolecule polymer or complex may be E3, E2, E1, a free ubiquitin, or a target substrate of E3. Here, the E2 enzyme may bind to the lysine at the 48th or 63rd amino acid residue of ubiquitin, and the E2 enzyme may be an E2-25K ubiquitin conjugating enzyme, or a ubiquitin conjugating enzyme complex Ucb13-MMS2.

In another embodiment, the present invention provides a linear polyfunctional multimeric biomolecule polymer comprised of a polyubiquitin scaffold and a biomolecule, wherein the polyubiquitin scaffold is formed by linking two or more ubiquitins, for example, through covalent bonds, and the biomolecule is each bound to the ubiquitin. The biomolecule is preferably each bound to the N-terminus of the ubiquitin. The initiator that initiates the formation of a linear multimeric biomolecule polymer may be E3, E2, E1, a free ubiquitin, or a substrate. In addition, the linear multimeric biomolecule polymer may be comprised of 2 to 20 biomolecules.

Hereinafter, the present invention is to be described in more detail through the following examples. These examples are only for describing the present invention in more detail, and it will be apparent to those of ordinary skill in the art that the scope of the present invention is not limited by these examples according to the gist of the present invention.

### MODE FOR CARRYING OUT THE INVENTION

### Preparation Example

### Preparation Example 1: Cloning, Expression and Purification of C-Terminal Fusion Protein

The gene encoding the UCT (Ubiquitin C-terminal Tag) (SEQ ID NO: 1) protein fusion used in the examples of the present invention was produced on request by Genscript Inc.

In order to prepare a Ub out gene construct that does not comprise a ubiquitin tag at the C-terminus, fast cloning system (Li C, Wen A, Shen B, Lu J, Huang Y, Chang Y (2011). Fast cloning: a highly simplified, purification-free, sequence- and ligation-independent PCR cloning method. BMC Biotechnol 11, 92.) was used. This method is a technology capable of linking genes (insertion, removal or substitution) in which if the PCR product is directly treated with only Dpn1 in the absence of a restriction enzyme and ligase, Dpn1 plays a role of a restriction enzyme and ligase through a mechanism that has not yet been identified along with polymerase. In this method, using a primer designed to overlap both terminus with Phusion polymerase (Thermo Fisher Scientific), PCR (95° C. for 3 minutes, 95° C. for 15 seconds—55° C. for 1 minute—72° C. for 1 minute/kb 18 times repeated, 72° C. for 5 minutes, 12° C. for 20 minutes) was carried out on all vectors except for the region to be deleted. Next, the resulting PCR product was subjected to Dpn1 treatment for 1 hour at 37° C., and transformed into E. coli DH5a (Novagen), and then the plasmid of interest was obtained. All gene constructs were identified by commercial DNA sequencing.

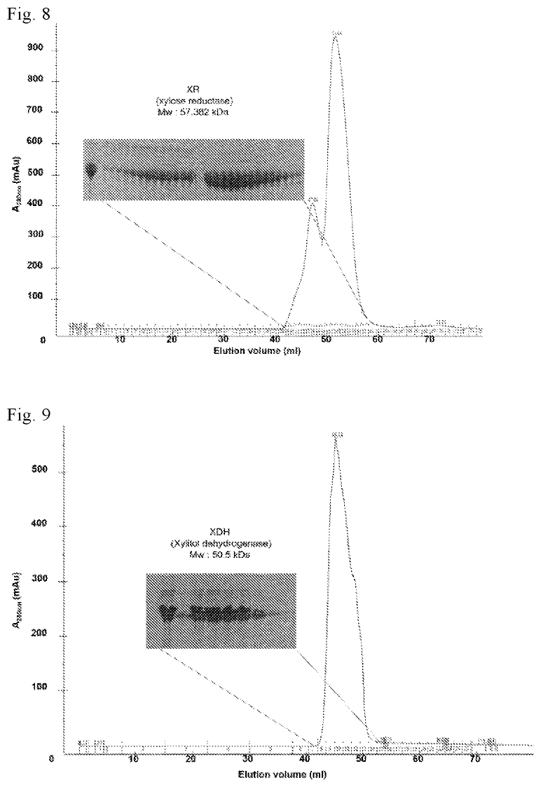

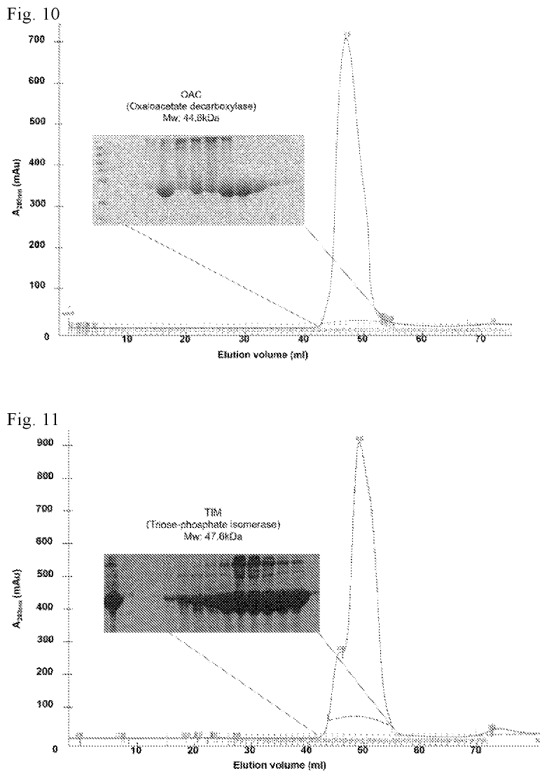

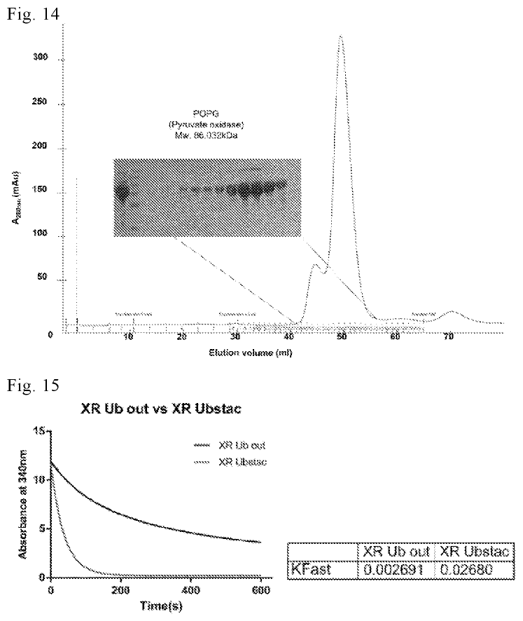

For overexpression of UCT fusion protein, each gene construct was transformed into E. coli BL21 DE3 (Novagen) (XR, TIM, ALD), Rosetta pLysS DE3 (Novagen) (XDH, OAC, POPG), Origami2 DE3 (Novagen) (FBP) strains. Cells comprising the protein expression plasmid (pET21a, Genscript) were incubated in LB medium (Miller) at 37° C. When the OD₆₀₀ value reached about 0.6, the protein expression was induced with 250 μM of isopropyl β-D-1-thiogalactopyranoside (isopropyl-beta-D-thiogalactopyranoside) (IPTG) at 16° C. for 20 hours. Next, after centrifugation (at 3,500 rpm at 4° C. for 15 minutes), cell pellet was resuspended in lysis buffer (20 mM Tris-HCl pH 8.0, 500 mM NaCl₂, 20 mM imidazole), and lysed by sonication (50% amplitude, pulse on 3 seconds-off 5 seconds, final 15 minutes). Then, the lysate was further centrifuged at 14,000 rpm at 4° C. for 30 minutes. The water soluble fraction of the protein comprising the N-terminal His-tag was purified by gel filtration chromatography using Superdex 75 pg gel filtration column 16/600 (GE Healthcare) pre-equilibrated with nickel affinity and FPLC buffer (Ni-NTA Agarose, QIAGEN, 20 mM Tris-HCl pH 8.0, 150 mM NaCl₂). All UCT proteins were concentrated to 100 μM for analysis of the enzyme activity. All target proteins were evaluated by SDS-PAGE. FIGS. 8 to 14 show the results of confirming the target proteins. The UCT fusion proteins used in the present invention are shown in Table 1 below.

TABLE 1

| | Molecular | |
|---|---|---|
| UCT protein fusion | weight (kDa) | SEQ ID NO |
| Xylose reductase (XR) | 57.382 | SEQ ID NO: 2 |
| Xylitol dehydrogenase (XDH) | 59.1 | SEQ ID NO: 3 |
| Oxaloacetate decarboxylase (OAC) | 44.6 | SEQ ID NO: 4 |
| Triose-phosphate isomerase (TIM) | 47.6 | SEQ ID NO: 5 |
| Aldolase (ALD) | 55.563 | SEQ ID NO: 6 |
| Fructose 1,6-bisphosphatase (FBP) | 49.3 | SEQ ID NO: 7 |
| Pyruvate oxidase (POPG) | 86.032 | SEQ ID NO: 8 |

### Preparation Example 2: Preparation of Ubstac Linear Construct

In the present invention, the reaction for preparing a linear polyfunctional multimeric fusion protein was designated as Ubstac reaction, respectively. The Ubstac reaction (a total volume of 50 μL) was carried out in the Ubstac buffer (25 mM HEPES (Sigma-aldrich), pH 7.5, 50 mM NaCl, 4 mM MgCl₂), and the Ubstac mixture for the Ubstac reaction (0.5 μM E1, M E2, 1 μM E3, 4 mM ATP) was added to the UCT protein fusion of the present invention to initiate the reaction. The ratio of protein used in the reaction was at a concentration of 10 uM to 20 uM UCT protein fusion per 1 uM E3 enzyme (a ratio of 1:10 to 1:20), which was a condition set for the purpose so that at least 10 fusion monomers form a linear polyfunctional multimer within 1 hour through the Ubstac reaction. The E1, E2 and E3 used in the present invention are as follows, respectively:

TABLE 2

| Category | Name | SEQ ID NO |
|---|---|---|
| E1 | Yeast UBE1 | SEQ ID NO: 9 |
| E2 | Ubch5a [Homo sapiens] | SEQ ID NO: 10 |
| | (UniProtKB - P51668) | |
| | Ubch7 [Homo sapiens] | SEQ ID NO: 11 |
| | (UniProtKB - P68036) | |
| | E2-25K [Homo sapiens] | SEQ ID NO: 12 |
| | (UniProtKB - P61086) | |
| | Ubc13 [Saccharomyces cerevisiae] | SEQ ID NO: 13 |
| | (UniProtKB - P52490) | |
| | MMS2 (UEV - Ubiquitin-conjugating | SEQ ID NO: 14 |
| | enzyme variant) | |
| E3 | RSP5 (UniProt ID. P39940) | SEQ ID NO: 15 |
| | DOA10 (UniProt ID. P40318) | SEQ ID NO: 16 |
| | MARCH5 (UniProt ID. Q9NX47) | SEQ ID NO: 17 |

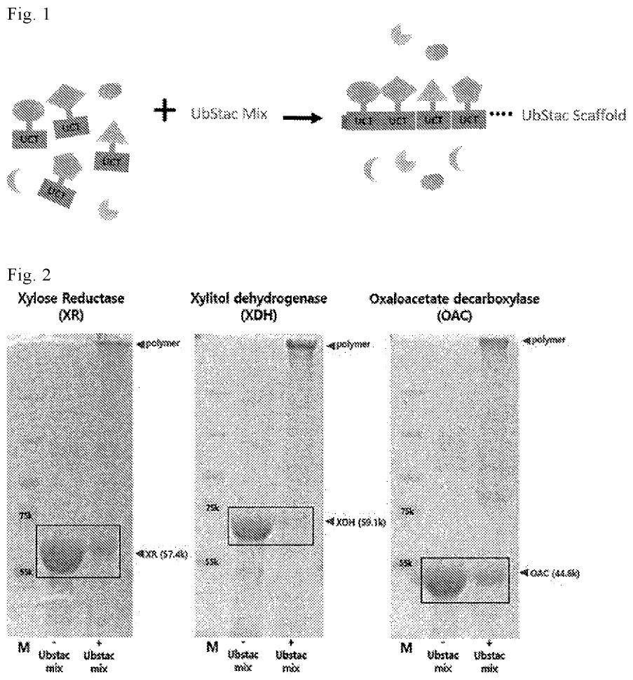

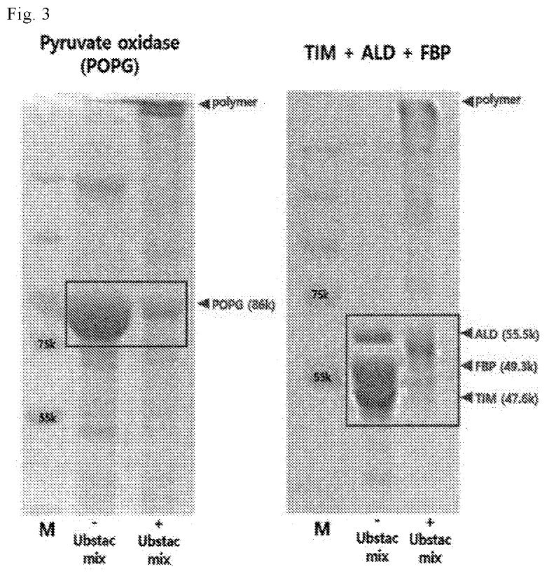

The Ubstac reaction was carried out by shaking at room temperature for 1 hour. FIG. 1 schematically shows the Ubstac reaction of the present invention, and FIGS. 2 and 3 show results of confirming the UCT fusion protein in a multimeric form formed by the Ubstac reaction.

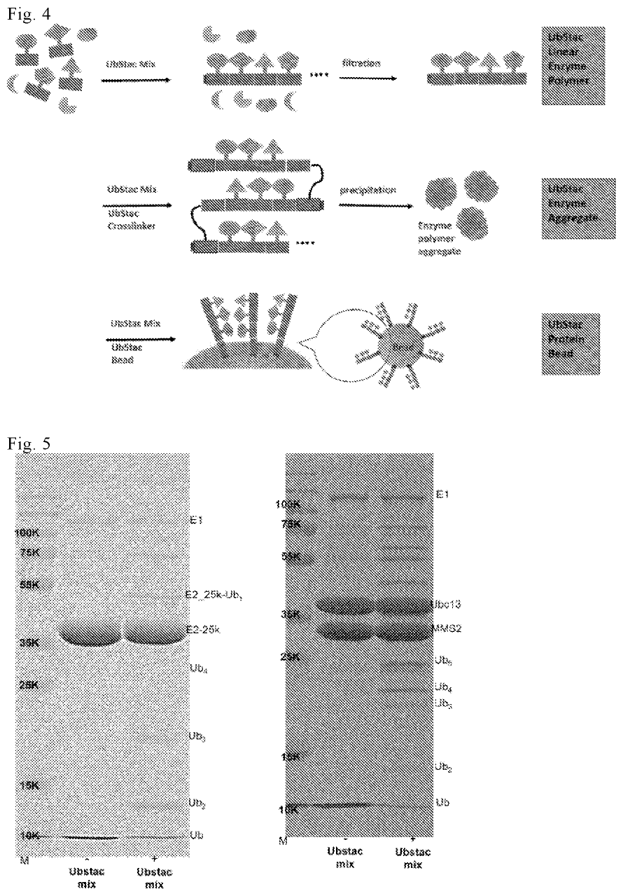

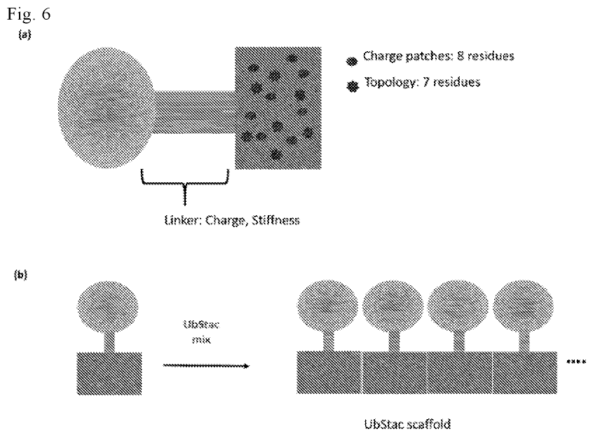

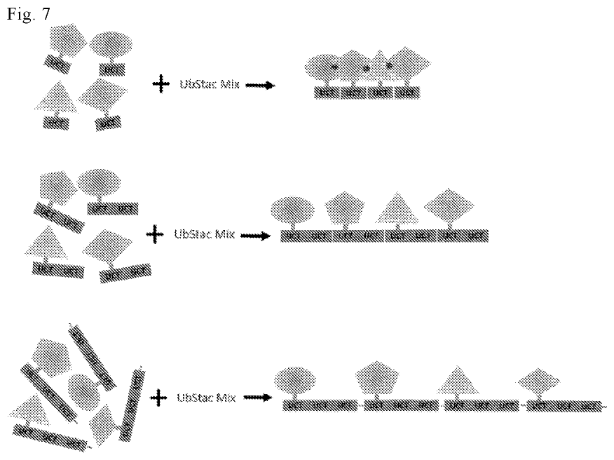

In FIG. 4, the first drawing schematically shows a process of preparing a Ubstac linear enzyme polymer by reacting a Ubstac mixture with a ubiquitin C terminal tagged enzyme as shown in FIG. 1 followed by filtration. The second drawing shows a process of preparing Ubstac enzyme aggregate by reacting the Ubstac mixture with a ubiquitin C-terminal tagged enzyme, followed by precipitation with a cross linker. The third drawing schematically shows a process of immobilizing the ubiquitin C terminal tagged protein onto a substrate or a bead.

### Preparation Example 3: Preparation of Ubstac Using Only E1-E2 (E2 Platform)

The E2-Ubstac was prepared by using E2-25K (GenBank ID-U58522.1) (human E2), Ucb13 (yeast E2)-MMS2 (GenBank ID-U66724.1) (yeast ubiquitin-conjugating enzyme variant) (GenBank ID-U66724.1). The recombinant DNA plasmid was requested to be synthesized by Genscript. The E2-Ubstac reaction (a total volume of 50 μL) was carried out under a condition of the E2-Ubstac buffer (50 mM Tris pH 8.0, 5 mM MgCl₂), and the E2-Ubstac mixture (1 uM E1, 10 uM E2, 4 mM ATP) was added to the free ubiquitin solution (20 μM) to initiate the reaction. The E2-Ubstac reaction was carried out by shaking at room temperature for 1 hour. The results are shown in FIG. 5.

### EXAMPLE

### Example 1: Analysis of Activity and Stability of Xylose Reductase (XR)

1-(1) Analysis of Activity of Xylose Reductase

The Ubstac reaction (a total volume of 50 μL) was carried out in the Ubstac buffer (25 mM HEPES pH 7.5, 50 mM NaCl, 4 mM MgCl₂), and the Ubstac mixture (0.5 μM E1, 5 μM E2, 1 μM E3, 4 mM ATP) was added to the XR protein solution to initiate the reaction. The Ubstac reaction was carried out by shaking at room temperature for 1 hour, and then the catalytic activity was analyzed. The catalytic activity of XR was analyzed by measuring the change in absorbance at 340 nm induced by NADH oxidation. The reaction for analysis of the catalytic activity was initiate by adding NADH (2 mM) to a mixture of XR (10 uM) and xylose (200 mM) in 100 mM NaCl buffer (pH 7.0) containing 1 mM MgCl₂ and 0.02% Tween-20. XR ub out was a sample in the form of a monomer that did not comprise a ubiquitin tag at the C-terminus of the XR, and did not form a polymer under the same Ubstac mixing condition. The statistical analysis was carried out using Prism 6 (GraphPad Software, Inc). The results are shown in FIG. 15. As shown in FIG. 15, the XR according to the present invention promoted the reduction of D-xylose to xylitol by using NADH as a co-substrate. Absorbance represents the amount of NADH in solution. The Ubstac polymer of the XR (red, lower curve) showed faster NADH consumption compared to the monomer form (black, upper curve). Both reactions contained the same amount of the monomers. Therefore, the increased reaction rate is solely dependent on the covalent bonds between the monomers. In this example, it was confirmed that the activity of the XR Ubstac polymer was increased by 10 times compared to the XR monomer without ubiquitin-tag (XR Ub out).

1-(2) Analysis of pH Stability of Xylose Reductase

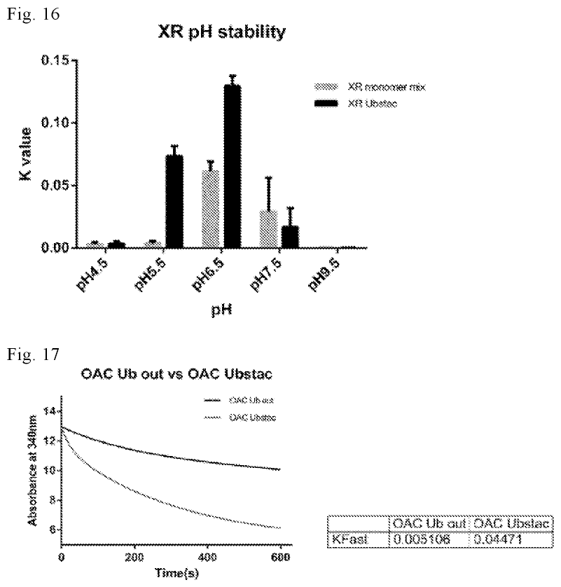

Both the XR monomer and the Ubstac polymer were treated for 30 minutes at the indicated pH before initiating the reaction with the addition of NADH and xylose. As shown in FIG. 16, at pH 5.5 and 6.5, the XR Ubstac polymer showed significantly enhanced stability compared to the XR monomer without ubiquitin-tag (XR Ub out). The results represent the average value of the three experiments.

### Example 2: Analysis of Activity and Stability of Oxaloacetate Decarboxylase (OAC)

2-(1) Analysis of Activity of OAC

OAC involved in gluconeogenesis is used to investigate liver damage in conjunction with AST-ALT. The Ubstac reaction (a total volume of 50 μL) was carried out in the Ubstac buffer (25 mM HEPES pH 7.5, 50 mM NaCl, 4 mM MgCl₂), and the Ubstac mixture (0.5 μM E1, 5 μM E2, 1 μM E3, 4 mM ATP) was added to the OAC protein solution to initiate the reaction. The Ubstac reaction was carried out by shaking at room temperature for 1 hour, and then the catalytic activity was analyzed. The analysis of OAC activity was based on the decrease in absorbance (340 nm) as NADH consumption proceeded under the following conditions: 45 mM TEA buffer pH 8.0, 0.45 mM MnCl₂, 2 mM NADH, 11 U of LDH, 5 μM OAC, 2.5 mM. The OAC Ubout was a sample in the form of a monomer that did not comprise a ubiquitin-tag at the C-terminus of the OAC, and did not form a polymer under the same Ubstac mixing condition. The statistical analysis was carried out using Prism 6 (GraphPad Software, Inc). The results are shown in FIG. 17. As shown in FIG. 17, as a result of comparing the activity of the monomer (OAC Ub out) without Ub at the C-terminus and the activity of the polymer (OAC Ubstac), the activity of the polymer was increased by 9 times. Absorbance represents the amount of NADH in solution. The Ubstac polymer of the OAC (red, lower curve) showed faster NADH consumption compared to the monomer form (black, upper curve). Both reactions contained the same amount of the monomers. Therefore, the increased reaction rate is solely dependent on the covalent bonds between the monomers. In this example, it was confirmed that the activity of the OAC Ubstac polymer was increased by 9 times compared to the OAC monomer without ubiquitin-tag (OAC Ub out).

2-(2) Analysis of Stability of OAC

Both the OAC monomer and the Ubstac polymer were treated for 30 minutes at the indicated pH before initiating the reaction with the addition of NADH and oxaloacetate. As shown in FIG. 18, at a low pH of pH 4.5 to 6.5, the OAC Ubstac polymer showed significantly enhanced pH stability compared to the OAC monomer without ubiquitin-tag (OAC Ub out). The results represent the average value of the three experiments.

### Example 3: Analysis of Activity and Stability of Xylitol Dehydrogenase (XDH)

3-(1) Analysis of Activity of XDH

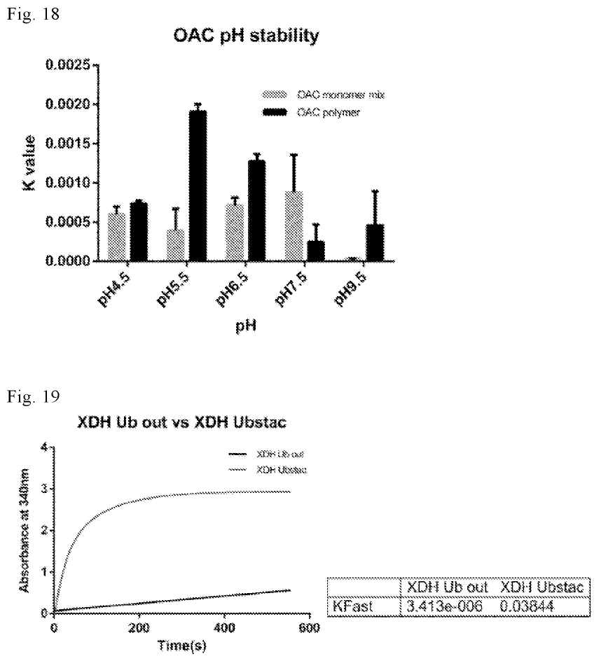

XDH is an enzyme belonging to the D-Xylose catabolism pathway, and is known to convert xylitol, a product of XR, into xylulose using NAD+. For analysis of activity of XDH, the Ubstac reaction (a total volume of 50 μL) was first carried out in the Ubstac buffer (25 mM HEPES pH 7.5, 50 mM NaCl, 4 mM MgCl₂), and the Ubstac mixture (0.5 μM E1, 5 μM E2, 1 M E3, 4 mM ATP) was added to the XDH protein solution to initiate the reaction. The Ubstac reaction was carried out by shaking at room temperature for 1 hour, and then the catalytic activity was analyzed. The activity of XDH was measured by monitoring NAD+ reduction at 340 nm. The reaction was initiated by adding NADH (2 mM) to a mixture of XDH (20 μM) and xylose (200 mM) in 100 mM NaCl buffer (pH 7.0) containing 1 mM MgCl₂ and 0.02% Tween-20. The XDH ub out was a sample in the form of a monomer that did not comprise a ubiquitin-tag at the C-terminus of the XDH, and did not form a polymer under the same Ubstac mixing condition. The statistical analysis was carried out using Prism 6 (GraphPad Software, Inc). The results are shown in FIG. 19. As shown in FIG. 19, at pH 5.5, the Ubstac polymer of the XDH (red, upper curve) showed higher NADH+ consumption rate compared to the monomer form (black, lower curve). Both reactions contained the same amount of the monomers. Therefore, the difference in activity is solely dependent on the covalent bonds between the monomers. In this example, it was confirmed that the activity of the XDH Ubstac polymer was increased by 10 times compared to the OAC monomer without ubiquitin-tag (OAC Ub out).

3-(2) Analysis of Stability of XDH

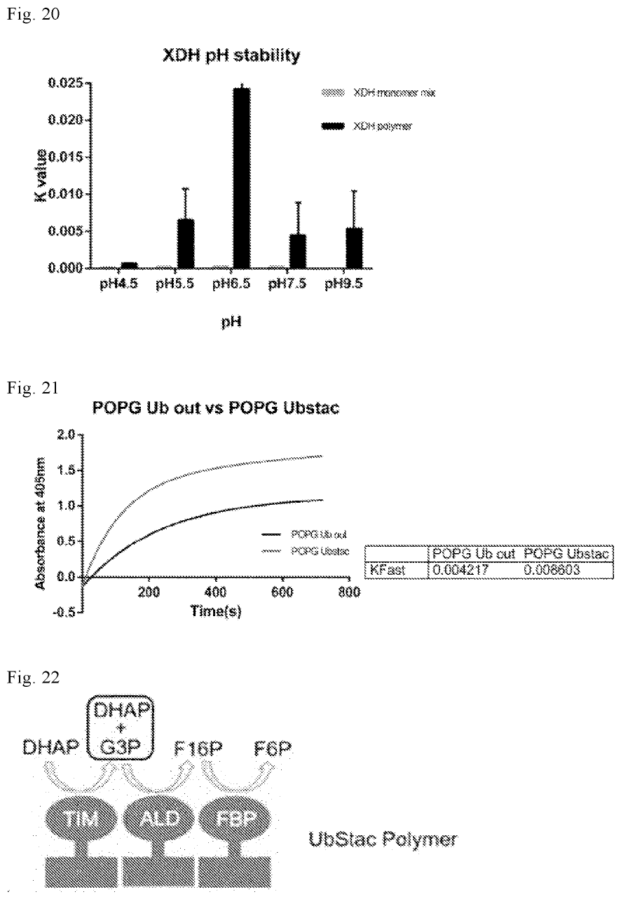

Both the XDH monomer and the Ubstac polymer were treated for 30 minutes at the indicated pH before initiating the reaction with the addition of NAD+ and xylitol. As shown in FIG. 20, at all measured pHs, the XR Ubstac polymer showed significantly increased pH stability compared to the OAC Ub out. The results represent the average value of the three experiments.

### Example 4: Analysis of Activity of Pyruvate Oxidase (POPG)

POPG is known to be used to investigate liver damage by detecting enzymes such as AST-ALT, an enzyme involved in the gluconeogenesis process. For analysis of activity of POPG, the Ubstac reaction (a total volume of 50 μL) was first carried out in the Ubstac buffer (25 mM HEPES pH 7.5, 50 mM NaCl, 4 mM MgCl₂), and the Ubstac mixture (0.5 uM E1, 5 uM E2, 1 uM E3, 4 mM ATP) was added to the POPG protein solution to initiate the reaction. The Ubstac reaction was carried out by shaking at room temperature for 1 hour, and then the catalytic activity was analyzed. In order to analyze the catalytic activity, the amount of H₂O₂ produced by the POPG oxidation process of pyruvate by ABTS was measured. The reaction was initiated by adding POPG (5 μM) to a mixture of pyruvate (100 mM), pyrophosphate (6 mM), ABTS (2,2′-azino-bis(3-ethylbenzothiazoline-6-sulfonic acid) (10 mM) and HRP (horseradish peroxidase) (0.2 U/mL) in sodium phosphate buffer. The POPG monomer (POPG ub out) was a sample in the form of a monomer that did not comprise a ubiquitin tag at the C-terminus of the POPG, and did not form a polymer under the same Ubstac mixing condition. The statistical analysis was carried out using Prism 6 (GraphPad Software, Inc). As shown in FIG. 21, at pH 5.5, the POPG (red, upper curve) showed higher activity compared to its monomer form (black, lower curve). Both reactions contained the same amount of the monomers. Therefore, the difference in activity is solely dependent on the covalent bonds between the monomers. In this particular example, the activity of the POPG UbStac polymer was increased by 2 times compared to the POPG monomer without ubiquitin-tag (POPG Ub out).

### Example 5: Analysis of Synergistic Effect of Ubiquitin Enzyme

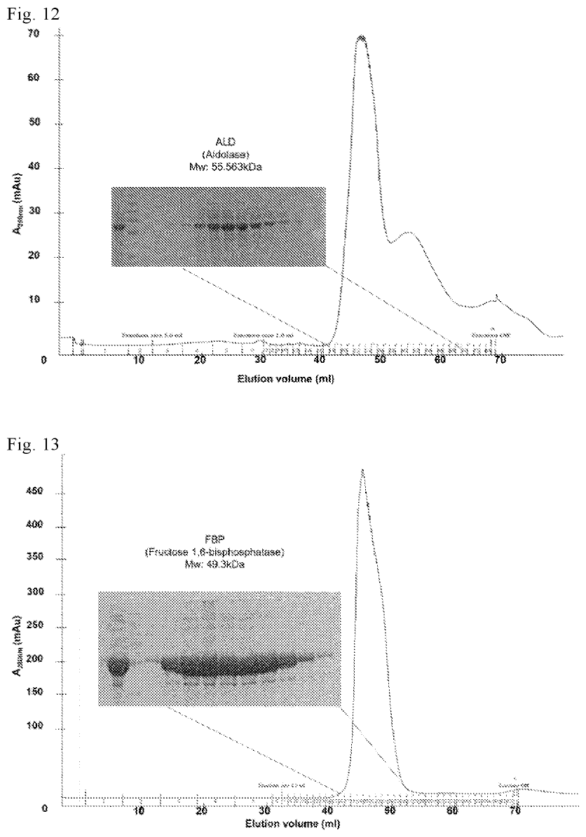

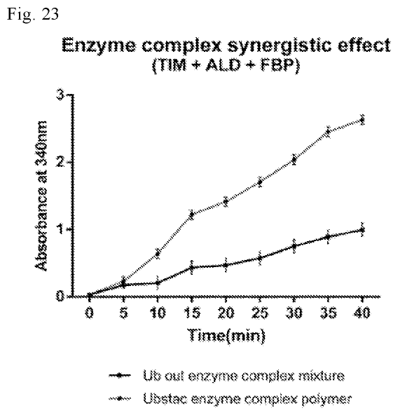

Triosephosphate isomerase (TIM), fructose bisphosphate aldolase (ALD) and fructose bisphosphatase (FBP) are known to form a cascade reaction for producing F6P as a final product from DHAP (dihydroxyacetone phosphate). The analysis of synergistic effect of the Ubstac enzyme was carried out by measuring Fructose-6-Phosphate (F6P), TIM product, ALD and FBP enzyme complex. F6P is isomerized to glucose-6-phosphate (G6P) by phosphoglucose isomerase (PGI), and the same amount of NAD+ as a substrate is modified by glucose-6-phosphate dehydrogenase (G6PDH). The present inventors determined the enzyme activity by measuring the amount of newly generated NADH by adding 2.5 mM of enzyme complex (dihydroxyacetone phosphate, DHAP), 20 U/mL analysis enzyme (PGI and G6PDH) and 2.5 mM NAD+ enzyme complex to a mixture of 4 uM TIM, ALD and FBP enzyme complex in a HEPES buffer condition (200 mM HEPES pH 7.5, 10 mM MgCl₂, 0.5 mM MnCl₂, 1 mM CaCl₂)) at 340 nm. The Ub out enzyme complex mixture was a sample in the form of a monomer that did not comprise a ubiquitin tag at the C-terminus of the enzyme, and did not form a polymer under the same Ubstac mixing condition. The statistical analysis was carried out using Prism 6 (GraphPad Software, Inc). At the indicated time point, the reaction was terminated, and the amount of F6P was measured using a phosphoglucose isomerase (PGI) that uses NAD+ to convert F6P into glucose-6-phosphate (G6P). Absorbance represents the amount of F6P. The Ubstac polymer of three different enzymes (red, upper curve) showed higher activity by five times than the monomeric enzyme mixture (black, lower curve). The results represent the average value of the three experiments. FIG. 23 shows the resulting Ubstac product of the structure in which three enzymes, TIM, ALD and FBP are bound, and FIG. 12 shows the synergistic effect by these enzymes.

### Example 9: Ubiquitin Multistage Labeling (Prosthetics) Method

First, a ubiquitin C-terminal tagged biomolecule was synthesized according to the Preparation Examples of the present invention. Next, a polymer (polyethylene glycol) comprising hydroxylamine was reacted with the above biomolecule. As a result, it was confirmed that the polymer was labeled by ubiquitin by oxime linking (the results are not shown).

### Example 10: Preparation of Protein a and Protein G Linear Multimer Polymer

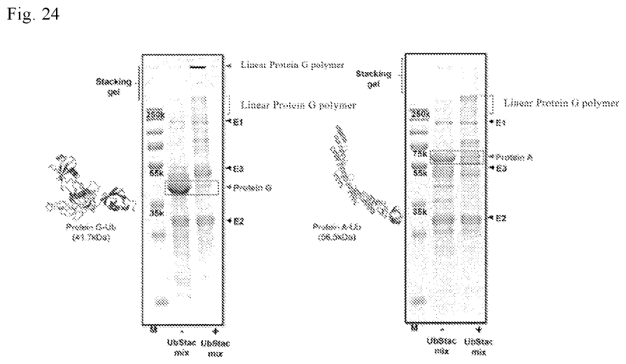

The Ubstac reaction (a total volume of 50 μL) was carried out in the Ubstac buffer (25 mM HEPES pH 7.5, 50 mM NaCl, 4 mM MgCl₂), and the Ubstac mixture (0.5 μM E1, 5 μM E2, 1 μM E3, 4 mM ATP) was added to the Protein A or Protein G solution to initiate the reaction. Recombinant DNA plasmids comprising sequences corresponding to Protein A (GenBank ID-AAB05743.1) and Protein G (CAA27638.1) were requested to be synthesized by Genscript. The Ubstac reaction was carried out by shaking at room temperature for 1 hour, and then SDS-PAGE was carried out. Compared with the sample without the Ubstac mixture, it was confirmed that the Protain A or Protein G monomer band was reduced in the sample to which the Ubstac mixture was added, and a band of high molecular weight (linear multimer polymer) newly appeared (see FIG. 24). It was confirmed that some linear multimer polymers did not pass through a stacking gel due to an increase in molecular weight of up to several hundreds kDa.

### Example 11: hGH in which C-Terminus of Glycine 76 of Ubiquitin C-Terminal Tag is Extended by Aspartate

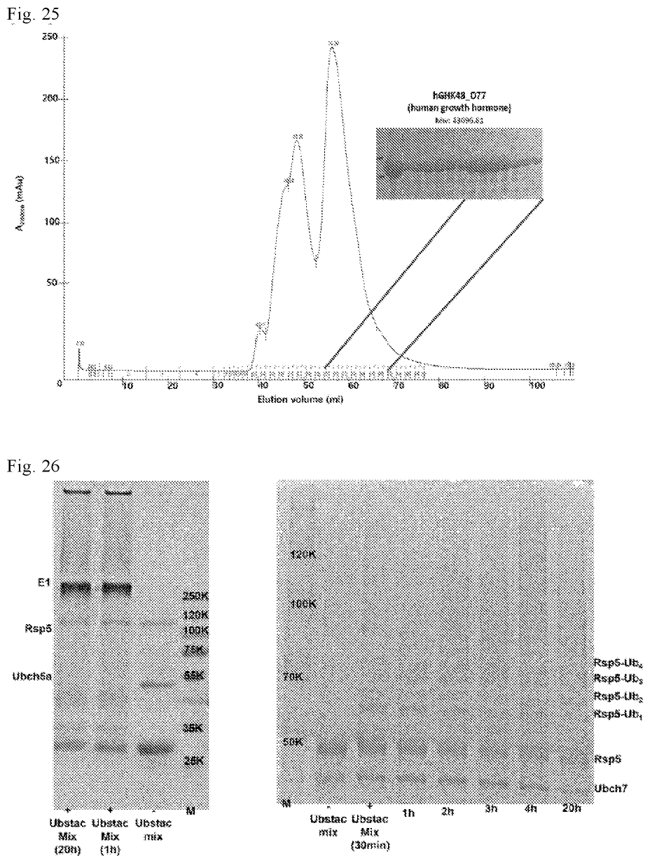

For overexpression of protein for UCT fusion drug, each gene construct was transformed into E. coli BL21 DE3 (Novagen) strain. In this example, hGH (SEQ ID NO: 18) was used as a protein. Cells comprising the protein expression plasmid (pET21a, Genscript) were incubated in LB medium (Miller) at 37° C. When the OD₆₀₀ value reached about 0.6, the protein expression was induced with 250 μM of isopropyl j-D-1-thiogalactopyranoside (isopropyl-beta-D-thiogalactopyranoside) (IPTG) at 16° C. for 20 hours. Next, after centrifugation (at 3,500 rpm at 4° C. for 15 minutes), cell pellet was resuspended in lysis buffer (20 mM Tris-HCl pH 8.0, 500 mM NaCl₂, 20 mM imidazole), and lysed by sonication (50% amplitude, pulse on 3 seconds-off 5 seconds, final 15 minutes). Then, the lysate was further centrifuged at 14,000 rpm at 4° C. for 30 minutes. The water soluble fraction of the protein comprising the N-terminal His-tag was purified by gel filtration chromatography using Superdex 75 pg gel filtration column 16/600 (GE Healthcare) pre-equilibrated with nickel affinity and FPLC buffer (Ni-NTA Agarose—QIAGEN, 20 mM Tris-HCl pH 8.0, 150 mM NaCl₂). The purified hGH was concentrated to 100 μM. All target proteins were evaluated by SDS-PAGE (see FIG. 25).

### Example 12: Preparation of Polyubiquitin Scaffold Originated from E3 (Rsp5)

The Ubstac reaction (a total volume of 50 μL) was carried out in the Ubstac buffer (25 mM HEPES pH 7.5, 50 mM NaCl, 4 mM MgCl₂), and the Ubstac mixture (0.5 μM E1, 5 μM E2 (Ubch5a or Ubch7), 1 uM E3, 4 mM ATP) was added to the protein solution to initiate the reaction. The Ubstac reaction was carried out by shaking at room temperature for 1 hour, and SDS-PAGE was carried out. It was confirmed that the amount of E3 was reduced in the sample to which Ubstac was added compared to the sample without the Ubstac mixture (see FIG. 26). This is because the band of E3 whose molecular mass was increased due to the formation of a polyubiquitin scaffold originated from E3 was shifted upwards. In the results of using Ubch7 E2, which is less reactive than Ubch5a E2, it was confirmed that the molecular weight was increased by adding ubiquitin one by one to Rsp5 over time.

### Example 13: Preparation of Polymer of hGH Expressed in Extended Form with Aspartate at C-Terminus of UCT Repeatedly Linked in Head-to-Tail Form

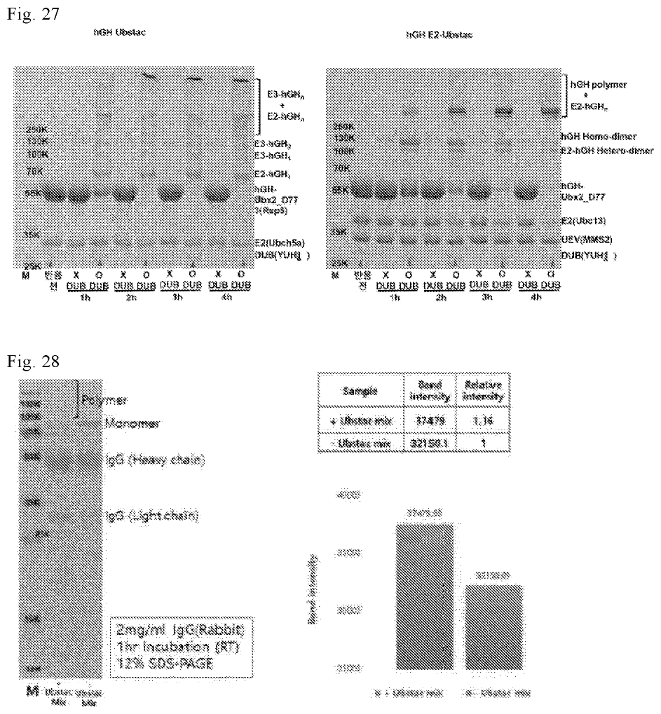

As shown in FIG. 27, the Ubstac reaction of hGH (SEQ ID NO: 18) was compared under the condition where DUB was present together and the condition where DUB was excluded. The hGH used at this time is one in which two ubiquitin tags are repeatedly connected at the C-terminus in a head-to-tail form, and the C-terminus of the ubiquitin tag is extended with aspartate. Therefore, if the aspartate at the C-terminus of the ubiquitin tag is not cleaved using DUB, the Ubstac reaction does not occur. The Ubstac reaction to confirm the polymer formation of hGH, a biomolecule for drug (a total volume of 50 μL) was carried out in the Ubstac buffer (25 mM HEPES pH 7.5, 50 mM NaCl, 4 mM MgCl₂), and the Ubstac mixture (1 μM E1, 5 μM E2 (ubch5a), 1 μM E3, 4 mM ATP) was added to 20 μM hGH protein solution to initiate the reaction. The E2-Ubstac reaction where E3 was excluded (a total volume of 50 μL) was carried out in the E2-Ubstac buffer (50 mM Tris pH 8.0, 5 mM MgCl₂), and the E2-Ubstac mixture (1 μM E1, 10 μM E2 (Ucb13-MMS2 complex), 4 mM ATP) was added to M hGH protein solution to initiate the reaction. In order to confirm the activity of DUB together, the reaction was carried out simultaneously under the condition where DUB (YUH1) was absent and the condition where DUB (YUH1) was present at a concentration of 2 μM, respectively. All reactions were carried out by shaking at room temperature for 1 to 4 hours, and then confirmed by SDS-PAGE. As shown in FIG. 27, it was confirmed that the polymer of hGH was formed only under the condition where DUB was present, and it was confirmed that the polymer was not formed because the aspartate at the C-terminus of hGH UCT was not cleaved under the condition where DUB was absent.

### Example 14: Preparation of Polymer of hGH Expressed in Extended Form with Aspartate at C-Terminus of UCT Repeatedly Linked in Head-to-Tail Form

The Ubstac reaction (a total volume of 50 μL) to prepare the Protein A polymer was carried out in the Ubstac buffer (25 mM HEPES pH 7.5, 50 mM NaCl, 4 mM MgCl₂). The Ubstac mixture (0.5 μM E1, 5 μM E2 (Ubch5a or Ubch7), 1 μM E3, 4 mM ATP) was added to the Protein A protein solution to initiate the reaction. The Ubstac reaction was carried out by shaking at room temperature for 1 hour, and then mixed in a 1:1 ratio with latex beads at 50% concentration, and then shaken at ambient temperature for 4 hours, and the reaction to immobilize the Protein A polymer on the beads was carried out. After the immobilization reaction, in order to remove the unimmobilized protein, washing was carried out three times with PBS buffer (10 mM Na₂HPO₄ pH 7.4, 1.8 mM KH₂PO₄, 137 mM NaCl, 2.7 mM KCl). After washing, the immunoglobulin G (IgG) obtained from human serum was added to the beads at a concentration of 2 mg/mL to analyze the binding activity of the Protein A polymer immobilized on the beads. The binding reaction was carried out by shaking at ambient temperature for 1 hour, and then washed three times with PBS buffer in the same manner as in the above washing method, and then confirmed by SDS-PAGE. As a result, it was confirmed that the binding activity of human derived IgG to the bead on which the protein A polymer was immobilized was increased by 15% or more compared to the bead on which the protein A monomer was immobilized by proceeding the same without adding the Ubstac mix (see FIG. 28).

### Example 15: Preparation of Linear Multivalent Biomolecule Polymer Bound to N-Terminus, C-Terminus, or Both N-Terminus and C-Terminus of Ubiquitin, Respectively

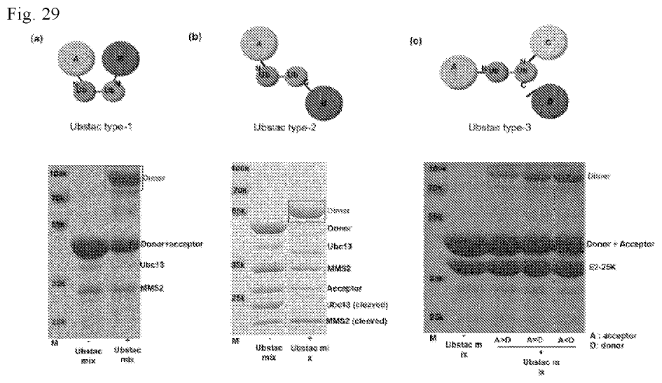

The formation of the Ubstac dimer was confirmed using the donor ubiquitin in which hGH (SEQ ID NO: 18) was bound to the N-terminus, the acceptor ubiquitin (FIG. 29 (a)) in which hGH was bound to the N-terminus, the acceptor ubiquitin (FIG. 29 (b)) in which hGH was bound to the C-terminus, and the acceptor ubiquitin (FIG. 29 (c)) in which sumo (SEQ ID NO: 19) and hGH were bound to the N-terminus and the C-terminus, respectively. The used acceptor ubiquitin is a form in which the leucine at the 73rd amino acid residue is substituted with proline, and other lysines except for the lysine at the 48th (FIG. 29 (c)) or the 63rd (FIG. 29 (a), (b)) amino acid residue are substituted with arginine, and the C-terminus is extended by aspartate or biomolecule (hGH). The Ubstac reaction (FIG. 29 (a), (b)) (a total volume of 50 μL) was carried out in the Ubstac buffer (25 mM HEPES pH 7.5, 50 mM NaCl, 4 mM MgCl₂), and the Ubstac mixture (1 uM E1, 5 uM E2 (Ubc13-MMS2 complex), 4 mM ATP) was added to a solution (a total ubiquitin concentration of 20 uM) in which 10 uM acceptor ubiquitin protein and donor ubiquitin protein were mixed to initiated the reaction. The Ubstac reaction (FIG. 29 (c)) was initiated by replacing E2 with E2-25K, not Ubc13-MMS2 complex, and the acceptor ubiquitin with a protein having only the lysine at the 48th amino acid residue other than the lysine at the 63rd amino acid residue under the same conditions as the above reaction. The Ubstac reaction was carried out by shaking at 27° C. for 4 hours, and then confirmed by SDS-PAGE. In the Ubstac reaction (FIG. 29 (b)), the acceptor ubiquitin in the Ub-hGH form in which His-sumo was cleaved using the SENP1 enzyme from a protein in the His-sumo-Ub-hGH form was used, and it was confirmed that at this time, the remaining SENP1 was included in the Ubstac reaction, and thus the donor hGH, Ubc13 and His-sumo of MMS2 were also cleaved together, and the band of dimer and E2 (Ubc13, MMS2) after reaction was shifted. As shown in FIG. 29, it was confirmed that the Ubstac dimer was formed in all forms in which the biomolecules were bound to the N-terminus, the C-terminus, or both of the N-terminus and the C-terminus, respectively. SEQ ID NO of proteins and the like used in this example are as follows: the donor ubiquitin in which hGH was bound to the N-terminus (SEQ ID NO: 20); the acceptor ubiquitin in which hGH was bound to the N-terminus (SEQ ID NO: 21); the acceptor ubiquitin in which hGH was bound to the C-terminus (SEQ ID NO: 22); and the acceptor ubiquitin in which SUMO was bound to the N-terminus and hGH was bound to the C-terminus (SEQ ID NO: 23).

### INDUSTRIAL AVAILABILITY

The present invention relates to a method for preparing a linear multimeric biomolecule polymer in which ubiquitin C-terminal tag (UCT)-biomolecule is a unit in vitro using the E1-E2-E3 system involved in the ubiquitin-proteasome proteolysis in vivo, and the linear multimeric biomolecule polymer prepared by this method. As described above, the linear multimeric biomolecule polymer of the present invention and a method for preparing the same can be widely used in industrial and medical fields requiring production of immobilized proteins, separation and purification of substances, and analysis.

1. A method for preparing a linear polyfunctional multimeric biomolecule, wherein the method comprises
(i) recombinantly expressing a biomolecule to which a ubiquitin C-terminal tag is fused or bound by a linker from a host cell including a prokaryotic cell or a eukaryotic cell, and
(ii) adding E1, E2 and E3 enzymes for ubiquitination, or E1 and E2 enzymes for ubiquitination to a cell lysate of the host cell and reacting them,
wherein the biomolecule is bound to a polyubiquitin scaffold formed of two or more covalently bonded ubiquitins, and the biomolecule comprises two or more binding moieties, each specific for different binding sites.

2. The method according to claim 1, wherein the E2 enzyme binds to the lysine at the 48th or 63rd amino acid residue of ubiquitin.

3. The method according to claim 2, wherein the E2 enzyme is an E2-25K ubiquitin conjugating enzyme.

4. The method according to claim 2, wherein the E2 enzyme is Ucb13-MMS2, a ubiquitin conjugating enzyme complex.

5. The method according to claim 1, wherein the biomolecule has active sites that specifically bind to other biomolecules, small molecule chemical compounds, or nanoparticles, and is an enzyme, a protein, a peptide, a polypeptide, an antibody, an antibody fragment, DNA or RNA.

6. The method according to claim 5, wherein the biomolecule is a protein A, protein G, lysin, endolysin, protease, hydrolase, oxidoreductase, lyase, affinity ligand, or receptor.

7. The method according to claim 1, wherein the biomolecule is selected from insulin, insulin analogue, glucagon, glucagon-like peptides (GLP-1 and the like), GLP-1/glucagon dual agonist, exendin-4, exendin-4 analogue, insulin secreting peptide and an analogue thereof, human growth hormone, growth hormone releasing hormone (GHRH), growth hormone releasing peptide, granulocyte colony stimulating factor (G-CSF), anti-obesity peptide, G-protein-coupled receptor, leptin, GIP (gastric inhibitory polypeptide), interleukins, interleukin receptors, interleukin binding proteins, interferons, interferon receptors, cytokine binding proteins, macrophage activator, macrophage peptide, B cell factor, T cell factor, suppressive factor of allergy, cell necrosis glycoprotein, immunotoxin, lymphotoxin, tumor necrosis factor (TNF), tumor inhibitory factor, metastasis growth factor, alpha-1 antitrypsin, albumin, α-lactalbumin, apolipoprotein-E, erythropoietin (EPO), high glycosylated erythropoietin, angiopoietins, hemoglobin, thrombin, thrombin receptor activating peptide, thrombomodulin, blood factors VII, VIIa, VIII, IX, and XIII, plasminogen activator, fibrin-binding peptide, urokinase, streptokinase, hirudin, protein C, C-reactive protein, renin inhibitor, collagenase inhibitor, superoxide dismutase, platelet derived growth factor, epithelial growth factor, epidermal growth factor, angiostatin, angiotensin, bone formation growth factor, bone formation promoting protein, calcitonin, atriopeptin, cartilage inducing factor, elcatonin, connective tissue activator, tissue factor pathway inhibitor, follicle stimulating hormone (FSH), luteinizing hormone (LH), luteinizing hormone releasing hormone (LHRH), nerve growth factors, parathyroid hormone (PTH), relaxin, secretin, somatomedin, adrenal cortical hormone, cholecystokinin, pancreatic polypeptide, gastrin releasing peptide, corticotropin releasing factor, thyroid stimulating hormone (TSH), autotaxin, lactoferrin, myostatin, receptor, receptor antagonist, fibroblast growth factor, adiponectin, interleukin receptor antagonist, cell surface antigen, virus derived vaccine antigen, monoclonal antibody, polyclonal antibody, and antibody fragments.

8. The method according to claim 1, wherein the recombinantly expressed biomolecule is one in which a C-terminal portion of the glycine at the 76th amino acid residue from the N-terminus of the ubiquitin, which is a ubiquitin C-terminal tag, is extended by 1 to 50 amino acids, and the method further comprises adding DUB (deubiquitinating enzyme) to the recombinantly expressed biomolecule before or after the reaction of step (ii).

9. The method according to claim 8, wherein the biomolecule is extended by aspartate, 6×His, chitin binding domain, GST, thrombin, FLAG tag.

10. The method according to claim 8, wherein the DUB is YUH1, YUH2, UCH-L1, UCH-L2 or UCH-L3.

11. The method according to claim 1, wherein the ubiquitin C-terminal tag is one in which other lysines except for one lysine at any position thereof are deleted or substituted with amino acids other than lysine.

12. The method according to claim 11, wherein all lysines of the ubiquitin except for the lysine at the 11th, 48th, or 63rd amino acid residue starting from the N-terminal Met1 of the ubiquitin are substituted with arginine.

13. The method according to claim 1, wherein the ubiquitin C-terminal tag is one in which two or more ubiquitins are repeatedly linked in a head-to-tail form.

14. The method according to claim 13, wherein the ubiquitin linked in the head-to-tail form is one in which the glycines at the 75th and 76th amino acid residues from the N-terminus are substituted with other amino acids including valine.

15. The method according to claim 13, wherein the leucine at the 73rd amino acid residue from the N-terminus of the ubiquitin is substituted with proline.

16. A linear polyfunctional multimeric biomolecule polymer comprised of a polyubiquitin scaffold and a biomolecule, wherein the linear polyfunctional biomolecule polymer comprises two or more binding moieties that are specific for different binding sites, and the polyubiquitin scaffold is formed of two or more covalently bonded ubiquitins; the biomolecule has active sites that specifically bind to other biomolecules, small molecule chemical compounds or nanoparticles or the like, and the biomolecule is bound to the N-terminus, the C-terminus, or both the N-terminus and the C-terminus of the ubiquitin.

17. The linear polyfunctional multimeric biomolecule polymer according to claim 16, wherein the linear multimeric biomolecule polymer is comprised of 2 to 4 biomolecules.

18. The linear polyfunctional multimeric biomolecule polymer according to claim 16, wherein the biomolecule is bound by a linker to the N-terminus, the C-terminus, or both the N-terminus and the C-terminus of the ubiquitin.

19. The linear polyfunctional multimeric biomolecule polymer according to claim 18, wherein the linker is a combination of 1 to 6 repeats of GGGGS or EAAAK.

20. The linear polyfunctional multimeric biomolecule polymer according to claim 16, wherein the biomolecule bound to the N-terminus of the ubiquitin is the distal end of the linear multimeric biomolecule polymer.

21. The linear polyfunctional multimeric biomolecule polymer according to claim 16, wherein the biomolecule bound to the C-terminus, the N-terminus, or both the C-terminus and the N-terminus of the ubiquitin is the proximal end of the linear multimeric biomolecule polymer.

22. The linear polyfunctional multimeric biomolecule polymer according to claim 16, wherein the polyubiquitin scaffold is formed by covalently linking a donor ubiquitin in which all lysines of the ubiquitin are substituted with arginine, and an acceptor ubiquitin in which all lysines of the ubiquitin except for the lysine at the 11th, 48th, or 63rd amino acid residue from the N-terminus are substituted with arginine.

23. The linear polyfunctional multimeric biomolecule polymer according to claim 16, wherein the leucine at the 73rd amino acid residue from the N-terminus of the ubiquitin is substituted with proline.

24. The linear polyfunctional multimeric biomolecule polymer according to claim 22, wherein the acceptor ubiquitin is extended by an aspartate, a 6×His tag, or a GST tag.

25. The linear polyfunctional multimeric biomolecule polymer according to claim 16, wherein the linear multimeric biomolecule polymer is comprised of 2 to 20 biomolecules.

26. The linear polyfunctional multimeric biomolecule polymer according to claim 16, wherein the biomolecule an enzyme, a protein, a peptide, a polypeptide, an antibody, an antibody fragment, DNA, or RNA.

27. The linear polyfunctional multimeric biomolecule polymer according to claim 18, wherein the biomolecule is a protein A, protein G, lysin, endolysin, protease, hydrolase, oxidoreductase, lyase, affinity ligand, or receptor.

28. The linear polyfunctional multimeric biomolecule polymer according to claim 16, wherein the biomolecule is selected from insulin, insulin analogue, glucagon, glucagon-like peptides (GLP-1 and the like), GLP-1/glucagon dual agonist, exendin-4, exendin-4 analogue, insulin secreting peptide and an analogue thereof, human growth hormone, growth hormone releasing hormone (GHRH), growth hormone releasing peptide, granulocyte colony stimulating factor (G-CSF), anti-obesity peptide, G-protein-coupled receptor, leptin, GIP (gastric inhibitory polypeptide), interleukins, interleukin receptors, interleukin binding proteins, interferons, interferon receptors, cytokine binding proteins, macrophage activator, macrophage peptide, B cell factor, T cell factor, suppressive factor of allergy, cell necrosis glycoprotein, immunotoxin, lymphotoxin, tumor necrosis factor (TNF), tumor inhibitory factor, metastasis growth factor, alpha-1 antitrypsin, albumin, α-lactalbumin, apolipoprotein-E, erythropoietin (EPO), high glycosylated erythropoietin, angiopoietins, hemoglobin, thrombin, thrombin receptor activating peptide, thrombomodulin, blood factors VII, VIIa, VIII, IX, and XIII, plasminogen activator, fibrin-binding peptide, urokinase, streptokinase, hirudin, protein C, C-reactive protein, renin inhibitor, collagenase inhibitor, superoxide dismutase, platelet derived growth factor, epithelial growth factor, epidermal growth factor, angiostatin, angiotensin, bone formation growth factor, bone formation promoting protein, calcitonin, atriopeptin, cartilage inducing factor, elcatonin, connective tissue activator, tissue factor pathway inhibitor, follicle stimulating hormone (FSH), luteinizing hormone (LH), luteinizing hormone releasing hormone (LHRH), nerve growth factors, parathyroid hormone (PTH), relaxin, secretin, somatomedin, adrenal cortical hormone, cholecystokinin, pancreatic polypeptide, gastrin releasing peptide, corticotropin releasing factor, thyroid stimulating hormone (TSH), autotaxin, lactoferrin, myostatin, receptor, receptor antagonist, fibroblast growth factor, adiponectin, interleukin receptor antagonist, cell surface antigen, virus derived vaccine antigen, monoclonal antibody, polyclonal antibody and antibody fragments.

29. The linear polyfunctional multimeric biomolecule polymer according to claim 16, wherein the linear multimeric biomolecule polymer is originated from E3, E2, E1, a free ubiquitin, or a substrate.

30. The linear polyfunctional multimeric biomolecule polymer according to claim 29, wherein the E3 is Rsp5, WWP1, nedd4 or XIAP, or a minimal catalytic domain thereof, and the E2 is Ubc7, Ubch5a, E2-25K, Ubc13-MMS2 complex, or a catalytic domain thereof.

31. The linear polyfunctional multimeric biomolecule polymer according to claim 30, wherein the free ubiquitin is one in which other lysines except for one lysine at any position thereof are deleted or substituted with amino acids other than lysine.

32. The linear polyfunctional multimeric biomolecule polymer according to claim 31, wherein the free ubiquitin is one in which all lysines except for the lysine at the 11th, 48th, or 63rd amino acid residue from the N-terminus thereof are substituted with arginine.

33. The linear polyfunctional multimeric biomolecule polymer according to claim 32, wherein the free ubiquitin is one in which a C-terminal portion of the glycine at the 76th amino acid residue from the N-terminus thereof is extended by 1 to 50 amino acids.

34. The linear polyfunctional multimeric biomolecule polymer according to claim 33, wherein the free ubiquitin is extended by aspartate, 6×His tag, or GST tag.

35. The linear polyfunctional multimeric biomolecule polymer according to claim 31, wherein the biomolecule is linked to the N-terminal Met1 of the free ubiquitin.

36. The linear polyfunctional multimeric biomolecule polymer according to claim 31, wherein E3, E2, E1, a free ubiquitin or a substrate is attached to one terminus of the biomolecule as an initiator.

37. The linear polyfunctional multimeric biomolecule polymer according to claim 29, wherein the substrate is a protein that comprises an amino acid sequence that recognizes E3 ligase and comprises one or more lysine to which ubiquitin is capable of binding.

38. The linear polyfunctional multimeric biomolecule polymer according to claim 37, wherein the protein has PPPY for Rsp5 or Nedd4-1,2.

39. The linear polyfunctional multimeric biomolecule polymer according to claim 31, wherein the ubiquitin is one in which other lysines except for one lysine at any position thereof are deleted or substituted with amino acids other than lysine.

40. The linear polyfunctional multimeric biomolecule polymer according to claim 16, wherein the biomolecule is a protein having a molecular weight of 40 to 100 kDa.

41. A polyubiquitin scaffold linker for site-specific binding of two or more biomolecules comprising two or more binding moieties, each specific for different binding sites, which includes
(i) recombinantly expressing a biomolecule to which a ubiquitin C-terminal tag, a ubiquitin, is fused or bound by a linker from a host cell, and
(ii) adding E1, E2 and E3 enzymes for ubiquitination, or E1 and E2 enzymes for ubiquitination to a cell lysate of the host cell and reacting them.


SEQUENCE LISTING (23 sequences)

SEQ ID NO: 1 - protein, 76 aa, Artificial Sequence [sequence omitted]

SEQ ID NO: 2 - protein, 506 aa, Artificial Sequence
  Xylose Reductase
MGHHHHHHSDQEAKPSTEDLGDKKEGEYIKLKVIGQDSSEIHFKVKMTTHLKKLKESYCQRQGVPMNSLRFLFEGQRIADNHTPKELGMEEEDVIEVYQEQTGGMPSIKLNSGYDMPAVGFGCWKVDVDTCSEQIYRAIKTGYRLFDGAEDYANEKLVGAGVKKAIDEGIVKREDLFLTSKLWNNYHHPDNVEKALNRTLSDLQVDYVDLFLIHFPVTFKFVPLEEKYPPGFYCGKGDNFDYEDVPILETWKALEKLVKAGKIRSIGVSNFPGALLLDLLRGATIKPSVLQVEHHPYLQQPRLIEFAQSRGIAVTAYSSFGPQSFVELNQGRALNTSPLFENETIKAIAAKHGKSPAQVLLRWSSQRGIAIIPKSNTVPHLLENKDVNSFDLDEQDFADIAKLDINLRFNDPWDWDKIPIFVGGHHHHHHMQIFVKTLTGKTITLEVEPSDTIENVKAKIQDKEGIPPDQQRLIFAGKQLEDGRTLSDYNIQKESTLHLVLRLRGG

SEQ ID NO: 3 - protein, 542 aa, Artificial Sequence
  Xylitol dehydrogenase
MGHHHHHHSDQEAKPSTEDLGDKKEGEYIKLKVIGQDSSEIHFKVKMTTHLKKLKESYCQRQGVPMNSLRFLFEGQRIADNHTPKELGMEEEDVIEVYQEQTGGMTANPSLVLNKIDDISFETYDAPEISEPTDVLVQVKKTGICGSDIHFYAHGRIGNFVLTKPMVLGHESAGTVVQVGKGVTSLKVGDNVAIEPGIPSRFSDEYKSGHYNLCPHMAFAATPNSKEGEPNPPGTLCKYFKSPEDFLVKLPDHVSLELGALVEPLSVGVHASKLGSVAFGDYVAVFGAGPVGLLAAAVAKTFGAKGVIVVDIFDNKLKMAKDIGAATHTFNSKTGGSEELIKAFGGNVPNVVLECTGAEPCIKLGVDAIAPGGRFVQVGNAAGPVSFPITVFAMKELTLFGSFRYGFNDYKTAVGIFDTNYQNGRENAPIDFEQLITHRYKFKDAIEAYDLVRAGKGAVKCLIDGPEMQIFVKTLTGKTITLEVEPSDTIENVKAKIQDKEGIPPDQQRLIFAGKQLEDGRTLSDYNIQKESTLHLVLRLRG

SEQ ID NO: 4 - protein, 407 aa, Artificial Sequence
  Oxaloacetate decarboxylase
MGHHHHHHSDQEAKPSTEDLGDKKEGEYIKLKVIGQDSSEIHFKVKMTTHLKKLKESYCQRQGVPMNSLRFLFEGQRIADNHTPKELGMEEEDVIEVYQEQTGGMYELGVVYRNIQRADRAAADGLAALGSATVHEAMGRVGLLKPYMRPIYAGKQVSGTAVTVLLQPGDNWMMHVAAEQIQPGDIVVAAVTAECTDGYFGDLLATSFQARGARALIIDAGVRDVKTLQEMDFPVWSKAISSKGTIKATLGSVNIPIVCAGMLVTPGDVIVADDDGVVCVPAARAVEVLAAAQKRESFEGEKRAKLASGVLGLDMYKMREPLEKAGLKYIDMQIFVKTLTGKTITLEVEPSDTIENVKAKIQDKEGIPPDQQRLIFAGKQLEDGRTLSDYNIQKESTLHLVLRLRGG

SEQ ID NO: 5 - protein, 430 aa, Artificial Sequence
  Triose-phosphate isomerase
MGHHHHHHSDQEAKPSTEDLGDKKEGEYIKLKVIGQDSSEIHFKVKMTTHLKKLKESYCQRQGVPMNSLRFLFEGQRIADNHTPKELGMEEEDVIEVYQEQTGGMRRVLVAGNWKMHKTPSEARVWFAELKRLLPPLQSEAAVLPAFPILPVAKEVLAETQVGYGAQDVSAHKEGAYTGEVSARMLSDLGCRYAIVGHSERRRYHGETDALVAEKAKRLLEEGITPILCVGEPLEVREKGEAVPYTLRQLRGSLEGVEPPGPEALVIAYEPVWAIGTGKNATPEDAEAMHQEIRKALSERYGEAFASRVRILYGGSVNPKNFADLLSMPNVDGGLVGGASLELESFLALLRIAGMQIFVKTLTGKTITLEVEPSDTIENVKAKIQDKEGIPPDQQRLIFAGKQLEDGRTLSDYNIQKESTLHLVLRLRGG

SEQ ID NO: 6 - protein, 496 aa, Artificial Sequence
  Aldolase
MGHHHHHHSDQEAKPSTEDLGDKKEGEYIKLKVIGQDSSEIHFKVKMTTHLKKLKESYCQRQGVPMNSLRFLFEGQRIADNHTPKELGMEEEDVIEVYQEQTGGMVTMPYVKNTKEILEKASKERYAIGAFNFNNMEFLQAILEAAEEEKAPVIVATSEGAIKYIGKGDIETGAKLAVEMVRTYAEKLSVPVALHLDHGRDFKVIMAAIKAGYSSVMIDASHLPFEENLRETKRIVEIAHAVGISVEAELGKLKGIEDNVVEKESVLVDPEEAKVFVKETEVDFLAPAIGTSHGAFKFKGEAQLDFERLKKVKEYTQIPLVLHGASMVPQDIVKLANEYGAELSGAKGVPEDMLKKAIELGINKINTDTDLRITFVAYLRKVLSEDKSQIDPRKIFKPVFEQVKEIVKERIRIFGSSGKAMQIFVKTLTGKTITLEVEPSDTIENVKAKIQDKEGIPPDQQRLIFAGKQLEDGRTLSDYNIQKESTLHLVLRLRGG

SEQ ID NO: 7 - protein, 437 aa, Artificial Sequence
  Fructose 1,6-bisphosphatase
MGHHHHHHSDQEAKPSTEDLGDKKEGEYIKLKVIGQDSSEIHFKVKMTTHLKKLKESYCQRQGVPMNSLRFLFEGQRIADNHTPKELGMEEEDVIEVYQEQTGGMLDRLDFSIKLLRKVGHLLMIHWGRVDNVEKKTGFKDIVTEIDREAQRMIVDEIRKFFPDENIMAEEGIFEKGDRLWIIDPIDGTINFVHGLPNFSISLAYVENGEVKLGVVHAPALNETLYAEEGSGAFFNGERIRVSENASLEECVGSTGSYVDFTGKFIERMEKRTRRIRILGSAALNAAYVGAGRVDFFVTWRINPWDIAAGLIIVKEAGGMVTDFSGKEANAFSKNFIFSNGLIHDEVVKVVNEVVEEIGGKMQIFVKTLTGKTITLEVEPSDTIENVKAKIQDKEGIPPDQQRLIFAGKQLEDGRTLSDYNIQKESTLHLVLRLRGG

SEQ ID NO: 8 - protein, 772 aa, Artificial Sequence
  Pyruvate oxidase
MGHHHHHHSDQEAKPSTEDLGDKKEGEYIKLKVIGQDSSEIHFKVKMTTHLKKLKESYCQRQGVPMNSLRFLFEGQRIADNHTPKELGMEEEDVIEVYQEQTGGMSDNKINIGLAVMKILESWGADTIYGIPSGTLSSLMDAMGEEENNVKFLQVKHEEVGAMAAVMQSKFGGNLGVTVGSGGPGASHLINGLYDAAMDNIPVVAILGSRPQRELNMDAFQELNQNPMYDHIAVYNRRVAYAEQLPKLVDEAARMAIAKRGVAVLEVPGDFAKVEIDNDQWYSSANSLRKYAPIAPAAQDIDAAVELLNNSKRPVIYAGIGTMGHGPAVQELARKIKAPVITTGKNFETFEWDFEALTGSTYRVGWKPANETILEADTVLFAGSNFPFSEVEGTFRNVDNFIQIDIDPAMLGKRHHADVAILGDAGLAIDEILNKVDAVEESAWWTANLKNIANWREYINMLETKEEGDLQFYQVYNAINNHADEDAIYSIDVGNSTQTSIRHLHMTPKNMWRTSPLFATMGIAIPGGLGAKNTYPDRQVWNIIGDGAFSMTYPDVVTNVRYNMPVINVVFSNTEYAFIKNKYEDTNKNLFGVDFTDVDYAKIAEAQGAKGFTVSRIEDMDRVMAEAVAANKAGHTVVIDCKITQDRPIPVETLKLDSKLYSEDEIKAYKERYEAANLVPFREYLEAEGLESKYIKMQIFVKTLTGKTITLEVEPSDTIENVKAKIQDKEGIPPDQQRLIFAGKQLEDGRTLSDYNIQKESTLHLVLRLRGG

SEQ ID NO: 9 - protein, 1024 aa, Artificial Sequence
  Yeast UBE1
MSSNNSGLSAAGEIDESLYSRQLYVLGKEAMLKMQTSNVLILGLKGLGVEIAKNVVLAGVKSMTVFDPEPVQLADLSTQFFLTEKDIGQKRGDVTRAKLAELNAYVPVNVLDSLDDVTQLSQFQVVVATDTVSLEDKVKINEFCHSSGIRFISSETRGLFGNTFVDLGDEFTVLDPTGEEPRTGMVSDIEPDGTVTMLDDNRHGLEDGNFVRFSEVEGLDKLNDGTLFKVEVLGPFAFRIGSVKEYGEYKKGGIFTEVKVPRKISFKSLKQQLSNPEFVFSDFAKFDRAAQLHLGFQALHQFAVRHNGELPRTMNDEDANELIKLVTDLSVQQPEVLGEGVDVNEDLIKELSYQARGDIPGVVAFFGGLVAQEVLKACSGKFTPLKQFMYFDSLESLPDPKNFPRNEKTTQPVNSRYDNQIAVFGLDFQKKIANSKVFLVGSGAIGCEMLKNWALLGLGSGSDGYIVVTDNDSIEKSNLNRQFLFRPKDVGKNKSEVAAEAVCAMNPDLKGKINAKIDKVGPETEEIFNDSFWESLDFVTNALDNVDARTYVDRRCVFYRKPLLESGTLGTKGNTQVIIPRLTESYSSSRDPPEKSIPLCTLRSFPNKIDHTIAWAKSLFQGYFTDSAENVNMYLTQPNFVEQTLKQSGDVKGVLESISDSLSSKPHNFEDCIKWARLEFEKKFNHDIKQLLFNFPKDAKTSNGEPFWSGAKRAPTPLEFDIYNNDHFHFVVAGASLRAYNYGIKSDDSNSKPNVDEYKSVIDHMIIPEFTPNANLKIQVNDDDPDPNANAANGSDEIDQLVSSLPDPSTLAGFKLEPVDFEKDDDTNHHIEFITACSNCRAQNYFIETADRQKTKFIAGRIIPAIATTTSLVTGLVNLELYKLIDNKTDIEQYKNGFVNLALPFFGFSEPIASPKGEYNNKKYDKIWDRFDIKGDIKLSDLIEHFEKDEGLEITMLSYGVSLLYASFFPPKKLKERLNLPITQLVKLVTKKDIPAHVSTMILEICADDKEGEDVEVPFITIHL

SEQ ID NO: 10 - protein, 147 aa, Artificial Sequence
  Ubch5a (Homo sapiens)
MALKRIQKELSDLQRDPPAHCSAGPVGDDLFHWQATIMGPPDSAYQGGVFFLTVHFPTDYPFKPPKIAFTTKIYHPNINSNGSICLDILRSQWSPALTVSKVLLSICSLLCDPNPDDPLVPDIAQIYKSDKEKYNRHAREWTQKYAM

SEQ ID NO: 11 - protein, 154 aa, Artificial Sequence [sequence omitted]

SEQ ID NO: 12 - protein, 200 aa, Artificial Sequence
  E2-25K (Homo sapiens)
MANIAVQRIKREFKEVLKSEETSKNQIKVDLVDENFTELRGEIAGPPDTPYEGGRYQLEIKIPETYPFNPPKVRFITKIWHPNISSVTGAICLDILKDQWAAAMTLRTVLLSLQALLAAAEPDDPQDAVVANQYKQNPEMFKQTARLWAHVYAGAPVSSPEYTKKIENLCAMGFDRNAVIVALSSKSWDVETATELLLSN

SEQ ID NO: 13 - protein, 153 aa, Artificial Sequence
  Ubc13 (Saccharomyces cerevisiae)
MASLPKRIIKETEKLVSDPVPGITAEPHDDNLRYFQVTIEGPEQSPYEDGIFELELYLPDDYPMEAPKVRFLTKIYHPNIDRLGRICLDVLKTNWSPALQIRTVLLSIQALLASPNPNDPLANDVAEDWIKNEQGAKAKAREWTKLYAKKKPE

SEQ ID NO: 14 - protein, 137 aa, Artificial Sequence
  MMS2 (UEV -Ubiquitin-conjugating enzyme variant) (Saccharomyces cerevisiae)
MSKVPRNFRLLEELEKGEKGFGPESCSYGLADSDDITMTKWNGTILGPPHSNHENRIYSLSIDCGPNYPDSPPKVTFISKINLPCVNPTTGEVQTDFHTLRDWKRAYTMETLLLDLRKEMATPANKKLRQPKEGETF

SEQ ID NO: 15 - protein, 807 aa, Artificial Sequence
  E3 RSP5
MPSSISVKLVAAESLYKRDVFRSPDPFAVLTIDGYQTKSTSAAKKTLNPYWNETFKFDDINENSILTIQVFDQKKFKKKDQGFLGVVNVRVGDVLGHLDEDTATSSGRPREETITRDLKKSNDGMAVSGRLIVVLSKLPSSSPHSQAPSGHTASSSTNTSSTTRTNGHSTSSTRNHSTSHPSRGTAQAVESTLQSGTTAATNTATTSHRSTNSTSSATRQYSSFEDQYGRLPPGWERRTDNFGRTYYVDHNTRTTTWKRPTLDQTEAERGQLNANTELERRQHRGRTLPGGSSDNSSVTVQVGGGSNIPPVNGAAAAAFAATGGTTSGLGELPSGWEQRFTPEGRAYFVDHNTRTTTWVDPRRQQYIRTYGPTNTTIQQQPVSQLGPLPSGWEMRLTNTARVYFVDHNTKTTTWDDPRLPSSLDQNVPQYKRDFRRKVIYFRSQPALRILPGQCHIKVRRKNIFEDAYQEIMRQTPEDLKKRLMIKFDGEEGLDYGGVSREFFFLLSHEMFNPFYCLFEYSAYDNYTIQINPNSGINPEHLNYFKFIGRVVGLGVFHRRFLDAFFVGALYKMMLRKKVVLQDMEGVDAEVYNSLNWMLENSIDGVLDLTFSADDERFGEVVTVDLKPDGRNIEVTDGNKKEYVELYTQWRIVDRVQEQFKAFMDGFNEIPEDLVTVFDERELELLIGGIAEIDIEDWKKHTDYRGYQESDEVIQWFWKCVSEWDNEQRARLLQFTTGTSRIPVNGFKDLQGSDGPRRFTIEKAGEVQQLPKSHTCFNRVDLPQYVDYDSMKQKLTLAVEETIGFGQE

SEQ ID NO: 16 - protein, 1319 aa, Artificial Sequence
  E3 DOA10
MDVDSDVNVSRLRDELHKVANEETDTATFNDDAPSGATCRICRGEATEDNPLFHPCKCRGSIKYMHESCLLEWVASKNIDISKPGADVKCDICHYPIQFKTIYAENMPEKIPFSLLLSKSILTFFEKARLALTIGLAAVLYIIGVPLVWNMFGKLYTMMLDGSSPYPGDFLKSLIYGYDQSATPELTTRAIFYQLLQNHSFTSLQFIMIVILHIALYFQYDMIVREDVFSKMVFHKIGPRLSPKDLKSRLKERFPMMDDRMVEYLAREMRAHDENRQEQGHDRLNMPAAAADNNNNVINPRNDNVPPQDPNDHRNFENLRHVDELDHDEATEEHENNDSDNSLPSGDDSSRILPGSSSDNEEDEEAEGQQQQQQPEEEADYRDHIEPNPIDMWANRRAQNEFDDLIAAQQNAINRPNAPVFIPPPAQNRAGNVDQDEQDFGAAVGVPPAQANPDDQGQGPLVINLKLKLLNVIAYFIIAVVFTAIYLAISYLFPTFIGFGLLKIYFGIFKVILRGLCHLYYLSGAHIAYNGLTKLVPKVDVAMSWISDHLIHDIIYLYNGYTENTMKHSIFIRALPALTTYLTSVSIVCASSNLVSRGYGRENGMSNPTRRLIFQILFALKCTFKVFTLFFIELAGFPILAGVMLDFSLFCPILASNSRMLWVPSICAIWPPFSLFVYWTIGTLYMYWFAKYIGMIRKNIIRPGVLFFIRSPEDPNIKILHDSLIHPMSIQLSRLCLSMFIYAIFIVLGFGFHTRIFFPFMLKSNLLSVPEAYKPTSIISWKFNTILLTLYFTKRILESSSYVKPLLERYWKTIFKLCSRKLRLSSFILGKDTPTERGHIVYRNLFYKYIAAKNAEWSNQELFTKPKTLEQAEELFGQVRDVHAYFVPDGVLMRVPSSDIVSRNYVQTMFVPVTKDDKLLKPLDLERIKERNKRAAGEFGYLDEQNTEYDQYYIVYVPPDFRLRYMTLLGLVWLFASILMLGVTFISQALINFVCSFGFLPVVKLLLGERNKVYVAWKELSDISYSYLNIYYVCVGSVCLSKIAKDILHFTEGQNTLDEHAVDENEVEEVEHDIPERDINNAPVNNINNVEEGQGIFMAIFNSIFDSMLVKYNLMVFIAIMIAVIRTMVSWVVLTDGILACYNYLTIRVFGNSSYTIGNSKWFKYDESLLFVVWIISSMVNFGTGYKSLKLFFRNRNTSKLNFLKTMALELFKQGFLHMVIYVLPIIILSLVFLRDVSTKQIIDISHGSRSFTLSLNESFPTWTRMQDIYFGLLIALESFTFFFQATVLFIQWFKSTVQNVKDEVYTKGRALENLPDES

SEQ ID NO: 17 - protein, 278 aa, Artificial Sequence
  E3 MARCH5
MPDQALQQMLDRSCWVCFATDEDDRTAEWVRPCRCRGSTKWVHQACLQRWVDEKQRGNSTARVACPQCNAEYLIVFPKLGPVVYVLDLADRLISKACPFAAAGIMVGSIYWTAVTYGAVTVMQVVGHKEGLDVMERADPLFLLIGLPTIPVMLILGKMIRWEDYVLRLWRKYSNKLQILNSIFPGIGCPVPRIPAEANPLADHVSATRILCGALVFPTIATIVGKLMFSSVNSNLQRTILGGIAFVAIKGAFKVYFKQQQYLRQAHRKILNYPEQEEA

SEQ ID NO: 18 - protein, 191 aa, Artificial Sequence [sequence omitted]

SEQ ID NO: 19 - protein, 96 aa, Artificial Sequence
  sumo
SDQEAKPSTEDLGDKKEGEYIKLKVIGQDSSEIHFKVKMTTHLKKLKESYCQRQGVPMNSLRFLFEGQRIADNHTPKELGMEEEDVIEVYQEQTGG

SEQ ID NO: 20 - protein, 371 aa, Artificial Sequence
  the donor ubiquitin in which hGH was bound to the N-terminus
MGHHHHHHSDQEAKPSTEDLGDKKEGEYIKLKVIGQDSSEIHFKVKMTTHLKKLKESYCQRQGVPMNSLRFLFEGQRIADNHTPKELGMEEEDVIEVYQEQTGGFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGFMQIFVRTLTGRTITLEVEPSDTIENVRARIQDREGIPPDQQRLIFAGRQLEDGRTLSDYNIQRESTLHLVLRLRGG

SEQ ID NO: 21 - protein, 355 aa, Artificial Sequence
  the acceptor ubiquitin in which hGH was bound to the N-terminus
MGHHHHHHSDQEAKPSTEDLGDKKEGEYIKLKVIGQDSSEIHFKVKMTTHLKKLKESYCQRQGVPMNSLRFLFEGQRIADNHTPKELGMEEEDVIEVYQEQTGGFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSGRTITLEVEPSDTIENVRARIQDREGIPPDQQRLIFAGRQLEDGRTLSDYNIQKESTLHLVLRPRGG

SEQ ID NO: 22 - protein, 268 aa, Artificial Sequence
  the acceptor ubiquitin in which hGH was bound to the C-terminus
MQIFVRTLTGRTITLEVEPSDTIENVRARIQDREGIPPDQQRLIFAGRQLEDGRTLSDYNIQKESTLHLVLRPRGGDFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 23 - protein, 372 aa, Artificial Sequence
  the acceptor ubiquitin in which SUMO was bound to the N-terminus and hGH was bound to the C-terminus
MGHHHHHHSDQEAKPSTEDLGDKKEGEYIKLKVIGQDSSEIHFKVKMTTHLKKLKESYCQRQGVPMNSLRFLFEGQRIADNHTPKELGMEEEDVIEVYQEQTGGMQIFVRTLTGRTITLEVEPSDTIENVRARIQDREGIPPDQQRLIFAGKQLEDGRTLSDYNIQRESTLHLVLRPRGGDFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF